(19) United States
(12) Patent Application Publication
(10) Pub. No.: US 2015/0239970 A1
(43) Pub. Date: Aug. 27, 2015

(54) Stable, Low Viscosity Antibody Formulation

(71) Applicant: MEDIMMUNE, LLC, Gaithersburg, MD (US)

(72) Inventors: Jared Bee, Gaithersburg, MD (US); Paul Santacroce, Gaithersburg, MD (US); Jiali Du, Gaithersburg, MD (US); Mariana Dimitrova, Medford, MA (US)

(21) Appl. No.: 14/437,585

(22) PCT Filed: Oct. 23, 2013
(86) PCT No.: PCT/US13/66313

§ 371 Date: Apr. 22, 2015

Related U.S. Application Data
(60) Provisional application No. 61/718,379, filed on Oct. 25, 2012.

(51) Int. Cl. C07K 16/24; A61K 39/395; A61K 9/00; A61K 47/22; A61K 47/18; A61K 47/26

(52) U.S. Cl. CPC C07K 16/248; A61K 47/183; A61K 47/26; A61K 9/0019; A61K 47/22; A61K 39/39591; C07K 2317/565; A61K 2039/505

(57) ABSTRACT

The present invention relates to a stable, low viscosity antibody formulation, wherein the formulation comprises a high concentration of anti-IL6 antibody. In some embodiments, the invention is directed to a stable, low viscosity antibody formulation comprising about 50 mg/mL to about 400 mg/mL of an anti-IL6 antibody, and arginine, wherein the antibody formulation is in an aqueous solution and has a viscosity of less than 20 cP at 23° C. Also provided are methods of making and methods of using such antibody formulations.

### FIELD OF THE INVENTION

The present invention relates to a stable, low viscosity antibody formulation, wherein the formulation comprises a high concentration of anti-IL6 antibody. In some embodiments, the invention is directed to a stable, low viscosity antibody formulation comprising about 50 mg/mL to about 400 mg/mL of an anti-IL6 antibody, and arginine, wherein the antibody formulation is in an aqueous solution and has a viscosity of less than 20 cP at 23° C. Also provided are methods of making and methods of using such antibody formulations.

### BACKGROUND OF THE INVENTION

Antibodies have been used in the treatment of various diseases and conditions due to their specificity of target recognition, thereby generating highly selective outcomes following systemic administration. While antibodies can have high specificity, the doses required to treat patients, particularly for a chronic condition, are typically large. New production and purification techniques have been developed to provide for large amounts of highly purified monoclonal antibodies to be produced. However, challenges still exist to stabilize these antibodies, and yet more challenges exist to provide the antibodies in a dosage form suitable for administration.

In order to treat subjects with large dosage amounts of a specific antibody, it is desirable to increase the concentration of the antibody in the dosage formulation. Higher concentration generally provide for smaller injection volume for injection. However, at higher concentrations, antibodies often exhibit characteristic problems including aggregation, precipitation, gelation, lowered stability, and/or increased viscosity.

Various methods have been proposed to overcome the challenges associated high concentration dosage forms. For example, to address the stability problem associated with high concentration antibody formulations, the antibody is often lyophilized, and then reconstituted shortly before administration. Reconstitution is generally not optimal, since it adds an additional step to the administration process, and could introduce contaminants to the formulation. Additionally, even reconstituted antibodies can suffer from aggregation and high viscosity.

Additional problems also exist for administering antibody formulations. In some instances, the antibody formulation is withdrawn from its container and diluted into an appropriate intravenous (IV) bag prior to administration. The prepared IV bag containing the antibody formulation is termed a ‘compounded sterile preparation’ (CSP). The CSP is often held for a short time before being administered to a subject. The CSP is usually visually inspected for signs of precipitation or contamination before they are infused into the patient. The desired time-frame for stability of a CSP is shorter than that of the antibody formulation, e.g., about 4 to 8 hours at room temperature and 24 to 36 hours under refrigerated conditions.

Placement of the antibody formulation into the IV bags can cause a reduction in stability. For antibody products, precipitation or particle formation can occur, and can be assessed by visual inspection of the IV solution, dose recovery by ultraviolet-visible absorbance, and stability with respect to formation of high molecular weight species (HMWS) by size exclusion chromatography (SEC). Potency can also be measured, and is generally assessed by a product-specific test.

Multiple potential sources can cause instability of the CSP. The colloidal and conformational stability of proteins are impacted by solution conditions such as ionic strength, pH and the presence of excipients such as disaccharides or amino acids. Surfactants are often added to protein formulations to protect against aggregation caused by interfacial stresses or to inhibit particle formation. A reduction in protein stability could occur if a formulation excipient is diluted below its necessary level. Additionally, exposure to the high ionic strength environment in saline IV bags may accelerate specific degradation pathways for some proteins.

Thus, a need exists to provide high concentration antibody formulations that can overcome many of these challenges. Additionally, a need exists for a method of adding an antibody formulation to an IV bag, wherein the antibody formulation does not degrade, precipitate, or otherwise loose efficacy during dilution.

### BRIEF SUMMARY OF THE INVENTION

The present invention is directed to stable, low viscosity, high concentration antibody formulations.

In some embodiments, the present invention is directed to a stable, low viscosity antibody formulation comprising: (a) about 150 mg/mL to about 400 mg/mL of an anti-IL-6 antibody, and (b) greater than about 150 mM arginine, wherein the antibody formulation is in an aqueous solution and has a viscosity of less than 20 cP at 23° C.

In some embodiments, the anti-IL-6 antibody comprises a variable heavy domain (VH) and a variable light domain (VL), wherein the VH domain comprises complementarity determining regions (CDRs) comprising SEQ ID NOs: 7, 8 and 9 and the VL domain comprises CDRs comprising SEQ ID NOs. 10, 11 and 12. In one embodiment, the anti-IL-6 antibody comprises SEQ ID NO:1 and SEQ ID NO:2.

In some embodiments, the antibody is stable at 2° C. to 8° C. for 12 months as determined by SEC HPLC.

In some embodiments, the viscosity of the antibody formulation is less than 14 cP at 23° C.

Various concentrations of arginine can be used. In some embodiments, the antibody formulation comprises greater than 200 mM arginine. In some embodiments, the antibody formulation comprises greater than 220 mM arginine. In some embodiments, the antibody formulation comprises 150 mM to 400 mM arginine.

Various other components can be included in the antibody formulation. In some embodiments, the antibody formulation further comprises a surfactant. In some embodiments, the surfactant is selected from the group consisting of polysorbate, pluronics, Brij, and other nonionic surfactants. In some embodiments, the surfactant is polysorbate 80. In some embodiments, the antibody formulation further comprises histidine. In some embodiments, the formulation is substantially free of trehalose. In some embodiments, the formulation is substantially free of a disaccharide. In some embodiments, the formulation is substantially free of a reducing sugar, a non-reducing sugar, or a sugar alcohol. In some embodiments the formulation is substantially free of an osmolyte.

In some embodiments, the formulation has an injection force of less than 8 N when passed through a 27 Gauge thin wall PFS needle (equivalent to a 25 Ga or 26 Ga needle). In some embodiments, the formulation has an osmolarity of between 300 and 450 mosm/kg.

The antibody in the antibody formulation can have various purity levels. In some embodiments, the antibody is greater than 90% (w/w) of total polypeptide composition of the antibody formulation.

In some embodiments, the invention is directed to a stable, low viscosity antibody formulation comprising: (a) about 150 mg/mL to about 400 mg/mL of an antibody, wherein the antibody comprises amino acid sequences of SEQ ID NOS:1 and 2, (b) about 150 mM to about 400 mM arginine, (c) about 0.01% to about 0.1% polysorbate 80, (d) about 20 mM to about 30 mM histidine, wherein the antibody formulation has a viscosity of less than 20 cP at 23° C.

In some embodiments, the invention is directed to a stable, low viscosity antibody formulation comprising: (a) about 150 mg/mL to about 400 mg/mL of an antibody, wherein the antibody comprises a variable heavy domain (VH) and a variable light domain (VL), wherein the VH domain comprises complementarity determining regions (CDRs) comprising SEQ ID NOs: 7, 8 and 9 and the VL domain comprises CDRs comprising SEQ ID NOs. 10, 11 and 12, (b) about 150 mM to about 400 mM arginine, (c) about 0.01% to about 0.1% polysorbate 80, and (d) about 20 mM to about 30 mM histidine, wherein the antibody formulation has a viscosity of less than 20 cP at 23° C.

In some embodiments, the invention is directed to a stable, low viscosity antibody formulation comprising: (a) about 150 mg/mL of an antibody, wherein the antibody comprises a variable heavy domain (VH) and a variable light domain (VL), wherein the VH domain comprises complementarity determining regions (CDRs) comprising SEQ ID NOs: 7, 8 and 9 and the VL domain comprises CDRs comprising SEQ ID NOs. 10, 11 and 12, (b) about 220 mM arginine, (c) about 0.07% polysorbate 80, and (d) about 25 mM histidine, wherein the antibody formulation has a viscosity of less than 20 cP at 23° C.

In some embodiments, the invention is directed to a stable, low viscosity antibody formulation comprising: (a) about 150 mg/mL of an antibody, wherein the antibody comprises a variable heavy domain (VH) and a variable light domain (VL), wherein the VH domain comprises complementarity determining regions (CDRs) comprising SEQ ID NOs: 7, 8 and 9 and the VL domain comprises CDRs comprising SEQ ID NOs. 10, 11 and 12, (b) about 150 mM arginine, (c) about 0.07% polysorbate 80, and (d) about 25 mM histidine, wherein the antibody formulation has a viscosity of less than 20 cP at 23° C.

In some embodiments, the invention is directed to A stable, low viscosity antibody formulation comprising: (a) about 50 mg/mL to about 200 mg/mL of an antibody, wherein the antibody comprises a variable heavy domain (VH) and a variable light domain (VL), wherein the VH domain comprises complementarity determining regions (CDRs) comprising SEQ ID NOs: 7, 8 and 9 and the VL domain comprises CDRs comprising SEQ ID NOs. 10, 11 and 12, (b) about 20 mM to about 400 mM arginine, (c) about 0.01% to about 0.1% polysorbate 80, (d) about 5 mM to about 100 mM histidine, and optionally (e) about 50 mM to about 400 mM trehalose, wherein the antibody formulation has a viscosity of less than 20 cP at 23° C.

In some embodiments, the invention is directed to a stable, low viscosity antibody formulation comprising: (a) about 50 mg/mL of an antibody, wherein the antibody comprises a variable heavy domain (VH) and a variable light domain (VL), wherein the VH domain comprises complementarity determining regions (CDRs) comprising SEQ ID NOs: 7, 8 and 9 and the VL domain comprises CDRs comprising SEQ ID NOs. 10, 11 and 12, (b) about 0.05% polysorbate 80, (c) about 25 mM histidine, and (d) about 225 mM trehalose, wherein the antibody formulation has a viscosity of less than 20 cP at 23° C.

In some embodiments, the invention is directed to A stable, low viscosity antibody formulation comprising: (a) about 100 mg/mL of an antibody, wherein the antibody comprises a variable heavy domain (VH) and a variable light domain (VL), wherein the VH domain comprises complementarity determining regions (CDRs) comprising SEQ ID NOs: 7, 8 and 9 and the VL domain comprises CDRs comprising SEQ ID NOs. 10, 11 and 12, (b) about 25 mM arginine, (c) about 0.07% polysorbate 80, (d) about 25 mM histidine, and (e) about 180 mM trehalose, wherein the antibody formulation has a viscosity of less than 20 cP at 23° C.

In some embodiments, the invention is directed to a method of treating pain associated with osteoarthritis in a subject, the method comprising administering the antibody formulations described herein. In some embodiments, the invention is directed to a method of treating pain associated with chronic lower back pain in a subject, the method comprising administering the antibody formulations described herein. In some embodiments, the invention is directed to a method of treating rheumatoid arthritis in a subject, the method comprising administering the antibody formulations described herein.

In some embodiments, the invention is directed to a method of making a stable, low viscosity antibody formulation, the method comprising: (a) concentrating an antibody to about 150 mg/mL to about 400 mg/mL, wherein the antibody comprises amino acid sequences of SEQ ID NOS:1 and 2; and (b) adding arginine to the antibody of (a) to achieve an antibody formulation having a concentration of arginine of greater than about 150 mM, wherein the antibody formulation of (b) is in an aqueous solution and has a viscosity of less than 20 cP at 23° C., and wherein the antibody formulation of (b) is stable at 2° C. to 8° C. for 12 months as determined by SEC HPLC.

### BRIEF DESCRIPTION OF THE FIGURES

FIG. 1 is a graph showing predicted stabilizing ability of various excipients for anti-IL6(YTE) antibody. It demonstrates that arginine is not predicted to be the most colloidally stabilizing excipient for this antibody. The most stabilizing excipients were predicted to be sucrose and trehalose while the least stabilizing were predicted to be NaCl and sodium sulfate.

FIG. 2 is a viscosity versus concentration curve for trehalose, sucrose, sorbitol and trehalose/NaCl.

FIG. 3 is a viscosity versus concentration curve for an antibody formulation with (i) 210 mM trehalose, (ii) 180 mM trehalose/25 mM arginine, (iii) 170 mM trehalose/50 mM arginine, (iv) 180 mM trehalose/90 mM arginine, (v) 150 mM arginine, or (vi) 220 mM arginine.

FIG. 4 is a viscosity versus concentration curve for an antibody formulation with (i) 210 mM trehalose, (ii) 180 mM trehalose/25 mM arginine, (iii) 170 mM trehalose/50 mM arginine, (iv) 180 mM trehalose/90 mM arginine, (v) 150 mM arginine, or (vi) 220 mM arginine.

FIG. 5 is a viscosity versus concentration curve for an antibody formulation with (i) 210 mM trehalose, (ii) 180 mM trehalose/25 mM arginine, (iii) 150 mM arginine, or (iv) 220 mM arginine.

FIG. 6 is a viscosity versus concentration curve for an antibody formulation with (i) 150 mM arginine, (ii) 220 mM arginine, or (iii) 75 mM trehalose/100 mM arginine.

FIG. 7 is a comparison of the viscosity of the antibody formulation at 150 mM arginine and 220 mM arginine.

FIG. 8 demonstrated the temperature dependence of viscosity for 100 mg/mL and 150 mg/mL antibody formulations containing various excipients.

FIG. 9 is the thermal stability profile for anti-IL6(YTE) antibody in 25 mM L-histidine/L-histidine hydrochloride monohydrate, 220 mM arginine hydrochloride, 0.07% (w/v) polysorbate 80, pH 6.0.

FIG. 10 is a photograph of the low dose sample of anti-IL6(YTE) antibody from an IV after mock-infusion through a 0.2 micron in-line filter and collection into a 3 cc glass vial (initial time point).

FIG. 11 is a photograph of the low dose sample of anti-IL6(YTE) antibody from an IV bag after mock-infusion through a 0.2 micron in-line filter and collection into a 3 cc glass vial, wherein the IV bag was treated with 0.012% w/v polysorbate 80.

### DETAILED DESCRIPTION OF THE INVENTION

It should be appreciated that the particular implementations shown and described herein are examples, and are not intended to otherwise limit the scope of the application in any way. It should also be appreciated that each of the embodiments and features of the invention described herein can be combined in any and all ways.

The published patents, patent applications, websites, company names, and scientific literature referred to herein are hereby incorporated by reference in their entirety to the same extent as if each was specifically and individually indicated to be incorporated by reference. Any conflict between any references cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the content clearly dictates otherwise.

Throughout the present disclosure, all expressions of percentage, ratio, and the like are “by weight” unless otherwise indicated. As used herein, “by weight” is synonymous with the term “by mass,” and indicates that a ratio or percentage defined herein is done according to weight rather than volume, thickness, or some other measure.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

Technical and scientific terms used herein have the meaning commonly understood by one of skill in the art to which the present application pertains, unless otherwise defined. Reference is made herein to various methodologies and materials known to those of skill in the art. Standard reference works setting forth the general principles of recombinant DNA technology include Sambrook et al., “Molecular Cloning: A Laboratory Manual,” 2nd Ed., Cold Spring Harbor Laboratory Press, New York (1989); Kaufman et al., Eds., “Handbook of Molecular and Cellular Methods in Biology in Medicine,” CRC Press, Boca Raton (1995); and McPherson, Ed., “Directed Mutagenesis: A Practical Approach,” IRL Press, Oxford (1991), the disclosures of each of which are incorporated by reference herein in their entireties.

The present invention is directed to stable, low viscosity antibody formulations. As described herein, the term “antibody formulation” refers to a composition comprising one or more antibody molecules. The term “antibody” in the present invention is not particularly limited. For clarity, an “antibody” is taken in its broadest sense and includes any immunoglobulin (Ig), active or desired variants thereof, and active or desirable fragments thereof (e.g., Fab fragments, camelid antibodies (single chain antibodies), and nanobodies). The term “antibody” can also refer to dimers or multimers. The antibody can be polyclonal or monoclonal and can be naturally-occurring or recombinantly-produced. Thus, human, non-human, humanized, and chimeric antibodies are all included with the term “antibody.” Typically the antibody is a monoclonal antibody of one of the following classes: IgG, IgE, IgM, IgD, and IgA; and more typically is an IgG or IgA.

An antibody of the invention can be from any animal origin including birds and mammals. In some embodiments, the antibody of the methods of the invention are human, murine (e.g., mouse and rat), donkey, sheep, rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins. See, e.g., U.S. Pat. No. 5,939,598 by Kucherlapati et al.

An antibody of the invention can include, e.g., native antibodies, intact monoclonal antibodies, polyclonal antibodies, multispecific antibodies (e.g., bispecific antibodies) formed from at least two intact antibodies, antibody fragments (e.g., antibody fragments that bind to and/or recognize one or more antigens), humanized antibodies, human antibodies (Jakobovits et al., Proc. Natl. Acad. Sci. USA 90:2551 (1993); Jakobovits et al., Nature 362:255-258 (1993); Bruggermann et al., Year in Immunol. 7:33 (1993); U.S. Pat. Nos. 5,591,669 and 5,545,807), antibodies and antibody fragments isolated from antibody phage libraries (McCafferty et al., Nature 348:552-554 (1990); Clackson et al., Nature 352:624-628 (1991); Marks et al., J. Mol. Biol. 222:581-597 (1991); Marks et al., Bio/Technology 10:779-783 (1992); Waterhouse et al., Nucl. Acids Res. 21:2265-2266 (1993)). An antibody purified by the method of the invention can be recombinantly fused to a heterologous polypeptide at the N- or C-terminus or chemically conjugated (including covalently and non-covalently conjugations) to polypeptides or other compositions. For example, an antibody purified by the method of the present invention can be recombinantly fused or conjugated to molecules useful as labels in detection assays and effector molecules such as heterologous polypeptides, drugs, or toxins. See, e.g., PCT publications WO 92/08495; WO 91/14438; WO 89/12624; U.S. Pat. No. 5,314,995; and EP 396,387.

In some embodiments, the antibody can be directed against one or more antigens, as is well known in the art. Examples of suitable anti-inflammatory antibodies include, but are not limited to, anti-TNF alpha antibodies such as adalimumab, infliximab, etanercept, golimumab, and certolizumab pegol; anti-IL1β antibodies such as canakinumab; anti-IL12/23 (p40) antibodies such as ustekinumab and briakinumab; and anti-IL2R antibodies, such as daclizumab. Examples of suitable anti-cancer antibodies include, but are not limited to, anti-BAFF antibodies such as belimumab; anti-CD20 antibodies such as rituximab; anti-CD22 antibodies such as epratuzumab; anti-CD25 antibodies such as daclizumab; anti-CD30 antibodies such as iratumumab, anti-CD33 antibodies such as gemtuzumab, anti-CD52 antibodies such as alemtuzumab; anti-CD152 antibodies such as ipilimumab; anti-EGFR antibodies such as cetuximab; anti-HER2 antibodies such as trastuzumab and pertuzumab; anti-IL6 antibodies such as siltuximab; and anti-VEGF antibodies such as bevacizumab; anti-IL6 receptor antibodies such as tocilizumab. In a particular embodiment, the antibody formulation comprises an anti-IL6 antibody.

In some embodiments, the antibody formulations comprise an anti-IL6 antibody, wherein the anti-IL6 antibody comprises a variable heavy domain (VH) and a variable light domain (VL), wherein the VH domain comprises complementarity determining regions (CDRs) comprising SEQ ID NOs: 7, 8 and 9 and the VL domain comprises CDRs comprising SEQ ID NOs. 10, 11 and 12.

Anti-IL6 Heavy Chain CDR1
SEQ ID NO: 7
SNYMI
Anti-IL6 Heavy Chain CDR2
SEQ ID NO: 8
DLYYYAGDTYYADSVKG
Anti-IL6 Heavy Chain CDR3
SEQ ID NO: 9
WADDHPPWIDL
Anti-IL6 Light Chain CDR1
SEQ ID NO: 10
RASQGISSWLA
Anti-IL6 Light Chain CDR2
SEQ ID NO: 11
KASTLES
Anti-IL6 Light Chain CDR3
SEQ ID NO: 12
QQSWLGGS

In some embodiments, the antibody formulation comprises an anti-IL6 antibody, wherein the anti-IL6 antibody comprises a VH domain and a VL domain comprising SEQ ID NOs; 5 and 6, respectively.

Anti-IL6 Variable Heavy Chain
SEQ ID NO: 5
EVQLVESGGGLVQPGGSLRLSCAASGFTISSNYMIWVRQAPGKGLEW
VSDLYYYAGDTYYADSVKGRFTMSRDISKNTVYLQMNSLRAEDTAVY
YCARWADDHPPWIDLWGRGTLVTVSS
Anti-IL6 Variable Light Chain
SEQ ID NO: 6
DIQMTQSPSTLSASVGDRVTITCRASQGISSWLAWYQQKPGKAPKVL
IYKASTLESGVPSRFSGSGSGTEFTLTISSLQPDDFATYYCQQSWLG
GSFGQGTKLEIK

In some embodiments, the antibody formulations comprise an anti-IL6 antibody as described by SEQ ID NOS. 3-4.

Anti-IL6 antibody Heavy Chain
SEQ ID NO: 3
EVQLVESGGGLVQPGGSLRLSCAASGFTISSNYMIWVRQAPGKGLEW
VSDLYYYAGDTYYADSVKGRFTMSRDISKNTVYLQMNSLRAEDTAVY
YCARWADDHPPWIDLWGRGTLVTVSSASTKGPSVFPLAPSSKSTSGG
TAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNK
ALPAPIEKTISKAKGQPREPQVYTLPPSRDELTKNQVSLTCLVKGFY
PSDIAVEWESNGQPENNYKTTPPVLDSDGSFFLYSKLTVDKSRWQQG
NVFSCSVMHEALHNHYTQKSLSLSPGK
Anti-IL6 antibody Light Chain
SEQ ID NO: 4
DIQMTQSPSTLSASVGDRVTITCRASQGISSWLAWYQQKPGKAPKVL
IYKASTLESGVPSRFSGSGSGTEFTLTISSLQPDDFATYYCQQSWLG
GSFGQGTKLEIKRTVAAPSVFIFPPSDEQLKSGTASVVCLLNNFYPR
EAKVQWKVDNALQSGNSQESVTEQDSKDSTYSLSSTLTLSKADYEKH
KVYACEVTHQGLSSPVTKSFNRGEC

In some embodiments, the antibody in the antibody formulation is a commercially available antibody, selected from the group consisting of adalimumab (Humira®, Abbott Laboratories), eculizumab (Soliris®, Alexion Pharmaceuticals), rituximab (Ritixan®, Roche/Biogen Idec/Chugai), infliximab (Remicade®, Johns on & Johnson/Schering-Plough/Tanabe), trastuzumab (Herceptin®, Roche/Chugai), bevacizumab (Avastin®, Chugai/Roche), palivizumab (Synagis®, Medlmmune/Abbott), alemtuzumab (Campath®, Genzyme), and motavizumab (Numax®, Medlmmune).

In some embodiments, the anti-IL6 antibody is a modified anti-IL6 antibody. For example, in some embodiments, the anti-IL6 antibody is anti-IL6(YTE) antibody, which contains three amino acid substitutions (M252Y/S254T/T256E) in the CH2 domain of the Fc domain, which have been shown to increase the serum half-life of Anti-IL6(YTE), as represented by SEQ ID NOS. 1-2.

anti-IL6(YTE) antibody Heavy Chain
SEQ ID NO: 1
EVQLVESGGGLVQPGGSLRLSCAASGFTISSNYMIWVRQAPGKGLEW
VSDLYYYAGDTYYADSVKGRFTMSRDISKNTVYLQMNSLRAEDTAVY
YCARWADDHPPWIDLWGRGTLVTVSSASTKGPSVFPLAPSSKSTSGG
TAALGCLVKDYFPEPVTVSWNSGALTSGVHTFPAVLQSSGLYSLSSV
VTVPSSSLGTQTYICNVNHKPSNTKVDKRVEPKSCDKTHTCPPCPAP
ELLGGPSVFLFPPKPKDTLYITREPEVTCVVVDVSHEDPEVKFNWYV
DGVEVHNAKTKPREEQYNSTYRVVSVLTVLHQDWLNGKEYKCKVSNK
ALPAPIEKTISKAKGQPREPQVYTLPPSREEMTKNQVSLTCLVKGFY
PSDIAVEWESNGQPENNYKTTPPVLDSDGSFFLYSKLTVDKSRWQQG
NVFSCSVMHEALHNHYTQKSLSLSPK
anti-IL6(YTE) antibody Light Chain
SEQ ID NO: 2
DIQMTQSPSTLSASVGDRVTITCRASQGISSWLAWYQQKPGKAPKVL
IYKASTLESGVPSRFSGSGSGTEFTLTISSLQPDDFATYYCQQSWLG
GSFGQGTKLEIKRTVAAPSVFIFPPSDEQLKSGTASVVCLLNNFYPR
EAKVQWKVDNALQSGNSQESVTEQDSKDSTYSLSSTLTLSKADYEKH
KVYACEVTHQGLSSPVTKSFNRGEC

See, e.g., Dall′Acqua et al., J. Immunol 169:5171-5180 (2002). Anti-IL6(YTE) antibody is a human IgG1κ monoclonal antibody with an overall molecular weight of approximately 148 kDa, containing one N-linked oligosaccharide attachment site in the Fc region at residue Asn-300. Anti-IL6(YTE) antibody is believed to block IL-6 receptor alpha ligand interactions and the subsequent functional events. The sequence of the anti-IL6(YTE) antibody can be found in SEQ ID NOS:1 and 2. Non-limiting examples for anti-IL-6 antibodies are also described in WO 2008/065378, WO 2010/088444, U.S. Pat. No. 8,198,414 and US Patent Appl. No. 20120034212 which are hereby incorporated by reference in their entireties.

For example, the nucleotide sequence of human IL-6 can be found in the GenBank database (see, e.g., Accession No. NM 000600.2). The amino acid sequence of human IL-6 can be found in the GenBank database (see, e.g., Accession No. P05231) and in U.S. patent application Ser. No. 10/496,793, filed Dec. 4, 2002, issued as U.S. Pat. No. 7,414,024 (see column 1); and U.S. patent application Ser. No. 12/470,753, filed May 22, 2009, issued as U.S. Pat. No. 7,833,755 (see column 19)(the amino acid sequence of human IL-6 is specifically incorporated herein by reference). Human IL-6 was also described in Hirano et al., Nature 324 (6092), 73-76 (1986). Each of these Assession numbers, patent applications, and journal articles are expressly incorporated by reference herein.

In one embodiment, an IL-6 polypeptide is human IL-6, an analog, derivative or a fragment thereof.

In some embodiments, the antibody formulation of the present invention comprises an anti-IL-6 antibody. Antibodies of the present invention specifically bind to an antigen of interest or a fragment thereof, and do not specifically bind to other antigens or fragments thereof. For example, an anti-I6 antibody will immunospecifically bind to an interleukin-6 polypeptide and does not specifically bind to other polypeptides. Preferably, antibodies or antibody fragments that immunospecifically bind to an IL-6 have a higher affinity to an IL-6 or a fragment of an IL-6 polypeptide when compared to the affinity to other polypeptides or fragments of other polypeptides. The affinity of an antibody is a measure of its bonding with a specific antigen at a single antigen-antibody site, and is in essence the summation of all the attractive and repulsive forces present in the interaction between the antigen-binding site of an antibody and a particular epitope. The affinity of an antibody to a particular antigen (e.g., an IL-6 polypeptide or fragment of an IL-6 polypeptide) may be expressed by the equilibrium constant K, defined by the equation K=[Ag Ab]/[Ag][Ab], which is the affinity of the antibody-combining site where [Ag] is the concentration of free antigen, [Ab] is the concentration of free antibody and [Ag Ab] is the concentration of the antigen-antibody complex. Where the antigen and antibody react strongly together there will be very little free antigen or free antibody, and hence the equilibrium constant or affinity of the antibody will be high. High affinity antibodies are found where there is a good fit between the antigen and the antibody (for a discussion regarding antibody affinity, see Sigal and Ron ed., 1994, Immunology and Inflammation—Basic Mechanisms and Clinical Consequences, McGraw-Hill, Inc. New York at pages 56-57; and Seymour et ah, 1995, Immunology—An Introduction for the Health Sciences, McGraw-Hill Book Company, Australia at pages 31-32). Preferably, antibodies or antibody fragments that immunospecifically bind to an IL-6 polypeptide or fragment thereof do not cross-react with other antigens. That is, antibodies or antibody fragments that immunospecifically bind to an IL-6 polypeptide or fragment thereof with a higher energy than to other polypeptides or fragments of other polypeptides (see, e.g., Paul ed., 1989, Fundamental Immunology, 2^{nd} ed., Raven Press, New York at pages 332-336 for a discussion regarding antibody specificity). Antibodies or antibody fragments that immunospecifically bind to an IL-6 polypeptide can be identified, for example, by immunoassays such as radioimmunoassays (RIAs), enzyme-linked immunosorbent assays (ELISAs), and BIAcore assays or other techniques known to those of skill in the art (see, e.g., Seymour et al, 1995, Immunology—An Introduction for the Health Sciences, McGraw-Hill Book Company, Australia at pages 33-41 for a discussion of various assays to determine antibody-antigen interactions in vivo). Antibodies or antibody fragments that immunospecifically bind to an IL-6 polypeptide or fragment thereof only antagonize an IL-6 polypeptide and do not significantly antagonize other activities.

As used herein, the term “analog” or “antibody analog” in the context of an antibody refers to a second antibody, ie., antibody analog, that possesses a similar or identical functions as the antibody, but does not necessarily comprise a similar or identical amino acid sequence of the antibody, or possess a similar or identical structure of the antibody. A antibody that has a similar amino acid sequence refers to an antibody analog that satisfies at least one of the following: (a) an antibody analog having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of the antibody; (b) an antibody analog encoded by a nucleotide sequence that hybridizes under stringent conditions to a nucleotide sequence encoding the antibody of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) an antibody analog encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding the antibody. An antibody analog with similar structure to the antibody refers to a proteinaceous agent that has a similar secondary, tertiary or quaternary structure to the antibody. The structure of an antibody analog or antibody can be determined by methods known to those skilled in the art, including but not limited to, peptide sequencing, X-ray crystallography, nuclear magnetic resonance, circular dichroism, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. One, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et ah, 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al, 1997, Nucleic Acids Res. 25:3389-3402. Alternatively, PSI-BLAST can be used to perform an iterated search which detects distant relationships between molecules (Id). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM 120 weight residue table, a gap length penalty of 12, and a gap penalty of 4 can be used.

In some embodiments, the antibody in the antibody formulation is purified prior to being added to the antibody formulation. The terms “isolate,” and “purify” refer to separating the antibody from an impurity or other contaminants in the composition which the antibody resides, e.g., a composition comprising host cell proteins. In some embodiments, at least 50%, 70%, 80%, 90%, 95%, 98%, 99%, 99.5%, or 99.9% (w/w) of an impurity is purified from the antibody. For example, in some embodiments, purification of an antibody, e.g. anti-IL6(YTE) antibody, would comprise separating the antibody from 99% (w/w) of the host cell proteins present originally in the composition.

In some embodiments, the terms “isolate,” and “purify” refer to separating an antibody, e.g. anti-IL6(YTE) antibody, from an impurity or other contaminants in the composition to an extent consistent with guidelines of a governmental organization, e.g., the World Health Organization or the United States Food and Drug Administration.

The antibody formulation of the present invention can be used for pharmaceutical purposes. Antibodies used in pharmaceutical applications generally must have a high level of purity, especially in regard to contaminants from the cell culture, including cellular protein contaminants, cellular DNA contaminants, viruses and other transmissible agents. See “WHO Requirements for the use of animal cells as in vitro substrates for the production of biologicals: Requirements for Biological Substances No. 50.” No. 878. Annex 1, 1998. In response to concerns about contaminants, The World Health Organization (WHO) established limits on the levels of various contaminants. For example, the WHO recommended a DNA limit of less than 10 ng per dose for protein products. Likewise, the United States Food and Drug Administration (FDA) set a DNA limit of less than or equal to 0.5 pg/mg protein. Thus, in some embodiments, the present invention is directed to antibody formulations meeting or exceeding contaminant limits as defined by one or more governmental organizations, e.g., the United States Food and Drug Administration and/or the World Health Organization.

In some embodiments, the antibody formulation described herein is pharmaceutically acceptable. “Pharmaceutically acceptable” refers to an antibody formulation that is, within the scope of sound medical judgment, suitable for contact with the tissues of human beings and animals without excessive toxicity or other complications commensurate with a reasonable benefit/risk ratio.

Purity of the antibody formulations can vary. In some embodiments, the therapeutic antibody of interest, e.g., Anti-IL6(YTE) antibody, is greater than 90% (wt/wt) of the total polypeptides present in the antibody formulation. In some embodiments, the therapeutic antibody of interest, e.g., anti-IL6(YTE), is greater than 95% (wt/wt), 98% (wt/wt), 99% (wt/wt), 99.5% (wt/wt) or 99.9% (wt/wt) of the total polypeptide present in the antibody formulation.

The concentration of the antibody in the antibody formulation can vary. In some embodiments, the antibody concentration in the antibody formulation is greater than about 20 mg/mL, greater than about 50 mg/mL, greater than about 75 mg/mL, greater than about 100 mg/mL, greater than about 125 mg/mL, greater than about 150 mg/mL, greater than about 175 mg/mL, or greater than about 200 mg/mL. In some embodiments, the antibody concentration in the antibody formulation is about 20 mg/mL to 300 mg/mL, about 50 mg/mL to about 250 mg/mL, about 75 mg/mL to about 200 mg/mL, about 100 mg/mL to about 175 mg/mL, about 125 mg/mL to about 175 mg/mL, about 50 mg/mL, about 100 mg/mL, or about 150 mg/mL.

The antibody formulation of the present invention can comprise arginine. Arginine is a conditionally non-essential amino acid that can be represented by the formula:

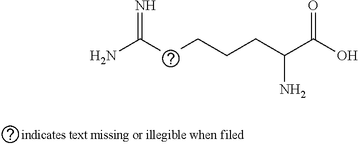 [SMILES: N=C(N)CCCCC(N)C(=O)O]

Arginine, as used herein, can include the free base form of arginine, as well as any and all salts thereof. In some embodiments, arginine includes a pharmaceutically acceptable salt thereof. For example, Arginine would include Arginine hydrochloride. Arginine, as used herein, also includes all enantiomers (e.g., L-arginine and D-arginine), and any combination of enantiomers (e.g., 50% L-arginine and 50% D-arginine; 90%-100% L-arginine and 10%-0% D-arginine, etc.). In some embodiments, the term “arginine” includes greater than 99% L-arginine and less than 1% D-arginine. In some embodiments, the term “arginine” includes an enantomerically pure L-arginine. In some embodiments, the arginine is a pharmaceutical grade arginine.

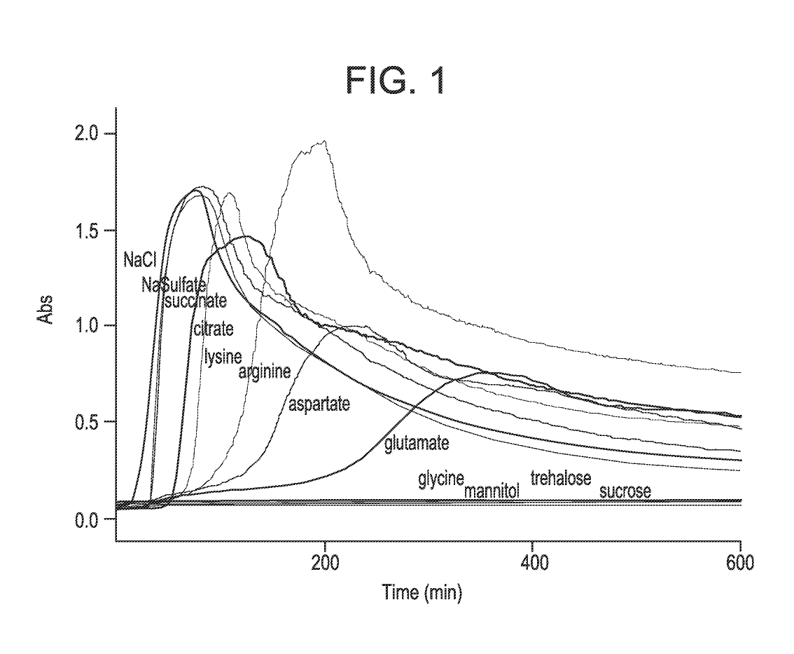

Arginine is expected to thermodynamically destabilize various antibodies, e.g., anti-IL6(YTE) antibodies. See, e.g., FIG. 1. One of skill in the art would expect increasing amounts of destabilizing agents, e.g. arginine, for a given protein, e.g. anti-IL6(YTE) antibodies, would have increased ability to alter protein structure from its native form, e.g., denature it. While not being bound by any particular theory, the inventors have found that even though increasing amounts of arginine in the antibody formulation did, in fact, decrease the melting temperature measured by DSC, the arginine actually provided a stabilizing effect, rather than a destabilizing effect, on the anti-IL6(YTE) antibody as measured by the SE-HPLC degradation rate upon storage. Thus, in some embodiments, high concentrations of arginine can be present in an antibody formulation and provide a stabilizing effect on the antibody in the formulation.

Various concentrations of arginine can be present in the antibody formulation. In some embodiments, the antibody formulation comprises greater than 20 mM arginine, greater than 25 mM arginine, greater than 50 mM arginine, greater than 75 mM arginine, greater than 100 mM arginine, greater than 125 mM arginine, greater than 150 mM arginine, greater than 175 mM arginine, greater than 200 mM arginine, 205 mM arginine, greater than 210 mM arginine, greater than 215 mM arginine, greater than 220 mM arginine, greater than 230 mM arginine, greater than 240 mM arginine, greater than 250 mM arginine, greater than 275 mM arginine, greater than 300 mM arginine, or greater than 350 mM arginine. In some embodiments, the antibody formulation comprises greater than 200 mM arginine. In some embodiments, the antibody formulation comprises greater than 220 mM arginine.

In some embodiments, the antibody formulation comprises up to 800 mM arginine, up to 700 mM arginine, up to 650 mM arginine, up to 600 mM arginine, up to 550 mM arginine, up to 500 mM arginine, up to 450 mM arginine, or up to 400 mM arginine.

In some embodiments, the antibody formulation comprises 25 mM to 600 mM arginine, 50 mM to 600 mM arginine, 75 mM to 600 mM arginine, 100 mM to 600 mM arginine, 125 mM to 500 mM arginine, 150 mM to 400 mM arginine, 175 mM to 400 mM arginine, 200 mM to 350 mM arginine. In some embodiments, the antibody formulation comprises 150 mM to 400 mM arginine.

As described herein, the antibody formulations comprising elevated concentrations of arginine have increased stability over time. Stability of the antibody in the antibody formulation can be determined by various means. In some embodiments, the antibody stability is determined by size exclusion chromatography (SEC). SEC separates analytes (e.g., macromolecules such as proteins and antibodies) on the basis of a combination of their hydrodynamic size, diffusion coefficient, and surface properties. Thus, for example, SEC can separate antibodies in their natural three-dimensional conformation from antibodies in various states of denaturation, and/or antibodies that have been degraded. In SEC, the stationary phase is generally composed of inert particles packed into a dense three-dimensional matrix within a glass or steel column. The mobile phase can be pure water, an aqueous buffer, an organic solvent, mixtures of these, or other solvents. The stationary-phase particles have small pores and/or channels which will only allow species below a certain size to enter. Large particles are therefore excluded from these pores and channels, but the smaller particles are removed from the flowing mobile phase. The time particles spend immobilized in the stationary-phase pores depends, in part, on how far into the pores they can penetrate. Their removal from the mobile phase flow causes them to take longer to elute from the column and results in a separation between the particles based on differences in their size.

In some embodiments, SEC is combined with an identification technique to identify or characterize proteins, or fragments thereof. Protein identification and characterization can be accomplished by various techniques, including but not limited chromatographic techniques, e.g., high-performance liquid chromatography (HPLC), immunoassays, electrophoresis, ultra-violet/visible/infrared spectroscopy, raman spectroscopy, surface enhanced raman spectroscopy, mass spectroscopy, gas chromatography, static light scattering (SLS), Fourier Transform Infrared Spectroscopy (FTIR), circular dichroism (CD), urea-induced protein unfolding techniques, intrinsic tryptophan fluorescence, differential scanning calorimetry, and/or ANS protein binding.

In some embodiments, protein identification is achieved by high-pressure liquid chromatography. Various instruments and apparatuses are known to those of skill in the art to perform HPLC. Generally HPLC involves loading a liquid solvent containing the protein of interest onto a separation column, in which the separation occurs. The HPLC separation column is filled with solid particles (e.g. silica, polymers, or sorbents), and the sample mixture is separated into compounds as it interacts with the column particles. HPLC separation is influenced by the liquid solvent's condition (e.g. pressure, temperature), chemical interactions between the sample mixture and the liquid solvent (e.g. hydrophobicity, protonation, etc.), and chemical interactions between the sample mixture and the solid particles packed inside of the separation column (e.g. ligand affinity, ion exchange, etc.).

In some embodiments, the SEC and protein identification occurs within the same apparatus, or simultaneously. For example, SEC and HPLC can be combined, often referred to as SE-HPLC.

By separating the various antibodies and antibody degradation products using known techniques such as those techniques identified herein, the stability of the antibody in the antibody formulation can be determined. As used herein, the term “stability” generally is related to maintaining the integrity or to minimizing the degradation, denaturation, aggregation or unfolding of a biologically active agent such as a protein, peptide or another bioactive macromolecule. As used herein, “improved stability” generally means that, under conditions known to result in degradation, denaturation, aggregation or unfolding, the protein (e.g., antibody such as anti-IL6(YTE)), peptide or another bioactive macromolecule of interest maintains greater stability compared to a control protein, peptide or another bioactive macromolecule. For example, the phrase “improved stability in the presence of arginine” would reflect that a protein of interest, e.g., anti-IL6(YTE) antibody, in the presence of arginine would have reduced amounts of degradation, denaturation, aggregation or unfolding of the anti-IL6(YTE) antibody relative to the same antibody which is not in the presence of arginine.

In some embodiments, stability refers to an antibody formulation having low to undetectable levels of aggregation. The phrase “low to undetectable levels of aggregation” as used herein refers to samples containing no more than 5%, no more than 4%, no more than 3%, no more than 2%, no more than 1% and no more than 0.5% aggregation by weight of protein as measured by high performance size exclusion chromatography (HPSEC), static light scattering (SLS), Fourier Transform Infrared Spectroscopy (FTIR), circular dichroism (CD), urea-induced protein unfolding techniques, intrinsic tryptophan fluorescence, differential scanning calorimetry, and 1-anilino-8-naphthalenesulfonic acid (ANS) protein binding techniques.

In some embodiments, the antibody formulation has low to undetectable levels of fragmentation. The term “low to undetectable levels of fragmentation” as used herein refers to samples containing equal to or more than 80%, 85%, 90%, 95%, 98% or 99% of the total protein, for example, in a single peak as determined by HPSEC, or in two peaks (e.g., heavy- and light-chains) (or as many peaks as there are subunits) by reduced Capillary Gel Electrophoresis (rCGE), representing the non-degraded antibody or a non-degraded fragment thereof, and containing no other single peaks having more than 5%, more than 4%, more than 3%, more than 2%, more than 1%, or more than 0.5% of the total protein in each. The term “reduced Capillary Gel Electrophoresis” as used herein refers to capillary gel electrophoresis under reducing conditions sufficient to reduce disulfide bonds in an antibody.

One of skill in the art will appreciate that stability of a protein is dependent on other features in addition to the composition of the formulation. For example, stability can be affected by temperature, pressure, humidity, and external forms of radiation. Thus, unless otherwise specified, stability referred to herein is considered to be measured at 2-8° C., one atmosphere pressure, 60% relative humidity, and normal background levels of radiation.

The term “stable” is relative and not absolute. Thus, for purposes herein, in some embodiments the antibody is stable if less than 20%, less than 15%, less than 10%, less than 5% or less than 2% of the antibody is degraded, denatured, aggregated or unfolded as determined by SEC HPLC when the antibody is stored at 2° C. to 8° C. for 6 months. In some embodiments, the antibody is stable if less than 20%, less than 15%, less than 10%, less than 5% or less than 2% of the antibody is degraded, denatured, aggregated or unfolded as determined by SEC HPLC when the antibody is stored at 2° C. to 8° C. for 12 months. In some embodiments, the antibody in the antibody formulation is stable if less than 20%, less than 15%, less than 10%, less than 5% or less than 2% of the antibody is degraded, denatured, aggregated or unfolded as determined by SEC HPLC when the antibody is stored at 2° C. to 8° C. for 18 months. In some embodiments, the antibody in the antibody formulation is stable if less than 20%, less than 15%, less than 10%, less than 5% or less than 2% of the antibody is degraded, denatured, aggregated or unfolded as determined by SEC HPLC when the antibody is stored at 2° C. to 8° C. for 24 months.

In some embodiments, the antibody is stable if less than 20%, less than 15%, less than 10%, less than 5% or less than 2% of the antibody is degraded, denatured, aggregated or unfolded as determined by SEC HPLC when the antibody is stored at 23° C. to 27° C. for 3 months. In some embodiments, the antibody is stable if less than 20%, less than 15%, less than 10%, less than 5% or less than 2% of the antibody is degraded, denatured, aggregated or unfolded as determined by SEC HPLC when the antibody is stored at 23° C. to 27° C. for 6 months. In some embodiments, the antibody is stable if less than 20%, less than 15%, less than 10%, less than 5% or less than 2% of the antibody is degraded, denatured, aggregated or unfolded as determined by SEC HPLC when the antibody is stored at 23° C. to 27° C. for 12 months. In some embodiments, the antibody is stable if less than 20%, less than 15%, less than 10%, less than 5% or less than 2% of the antibody is degraded, denatured, aggregated or unfolded as determined by SEC HPLC when the antibody is stored at 23° C. to 27° C. for 24 months.

In some embodiments the antibody is stable if less than 6%, less than 4%, less than 3%, less than 2% or less than 1% of the antibody is degraded, denatured, aggregated or unfolded per month as determined by SEC HPLC when the antibody is stored at 40° C. In some embodiments the antibody is stable if less than 6%, less than 4%, less than 3%, less than 2% or less than 1% of the antibody is degraded, denatured, aggregated or unfolded per month as determined by SEC HPLC when the antibody is stored at 5° C.

In some embodiments, the antibody formulations of the present invention can be considered stable if the antibody exhibit very little to no loss of the binding activity of the antibody (including antibody fragments thereof) of the formulation compared to a reference antibody as measured by antibody binding assays know to those in the art, such as, e.g., ELISAs, etc., over a period of 8 weeks, 4 months, 6 months, 9 months, 12 months or 24 months.

The antibody formulations described herein can have various viscosities. Methods of measuring viscosity of antibody formulations are known to those in the art, and can include, e.g., a rheometer (e.g., Anton Paar MCR301 Rheometer with either a 50 mm, 40 mm or 20 mm cone accessory). In some embodiments of the present invention, the viscosities were reported at a high shear limit of 1000 per second shear rate. In some embodiments, the antibody formulation has a viscosity of less than 20 cP, less than 18 cP, less than 15 cP, less than 13 cP, or less than 11 cP. In some embodiments, the antibody formulation has a viscosity of less than 14 cP. One of skill in the art will appreciate that viscosity is dependent on temperature, thus, unless otherwise specified, the viscosities provided herein are measured at 23° C. unless otherwise specified. In some embodiments, the viscosity of the antibody formulation is less than 14 cP at 23° C.

The term “injection force” is the amount of pressure (in Newtons) required to pass the antibody formulation through a needle. The injection force is correlated with the amount of resistance provided by the antibody formulation when administering the antibody formulation to a subject. The injection force will be dependent on the gauge of the administering needle, as well as temperature. In some embodiments, the antibody formulation has an injection force of less than 15 N, 12 N, 10N, or 8 N when passed through a 27 Ga thin wall PFS needle such as defined in the International Organization for Standardization (ISO) document “Stainless steel needle tubing for the manufacture of medical devices” (ISO 9626:1991) and manufactured by BD Medical, Pharmaceutical Systems (Franklin Lakes, N.J.). In some embodiments, the antibody formulation has an injection force of less than 15 N, 12 N, 10N, or 8 N when passed through a 25 or 26 Gauge needle

The antibody formulations can have different osmolarity concentrations. Methods of measuring osmolarity of antibody formulations are known to those in the art, and can include, e.g., an osmometer (e.g., an Advanced Instrument Inc 2020 freezing point depression osmometer). In some embodiments, the formulation has an osmolarity of between 200 and 600 mosm/kg, between 260 and 500 mosm/kg, or between 300 and 450 mosm/kg. In some embodiments, the formulation does not comprise an osmolyte.

The antibody formulation of the present invention can have various pH levels. In some embodiments, the pH of the antibody formulation is between 4 and 7, between 4.5 and 6.5, or between 5 and 6. In some embodiments, the pH of the antibody formulation is 6.0. Various means may be utilized in achieving the desired pH level, including, but not limited to the addition of the appropriate buffer.

Various other components can be included in the antibody formulation. In some embodiments, the antibody formulation can comprise a buffer (e.g. acetate, phosphate or citrate buffer), a surfactant (e.g. polysorbate), and/or a stabilizer agent (e.g. human albumin), etc. In some embodiments, the antibody formulation can comprise pharmaceutically acceptable carriers, including, e.g., ion exchangers, alumina, aluminum stearate, lecithin, serum proteins, such as human serum albumin, buffer substances such as phosphates, sucrose, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, polyethylene-polyoxypropylene-block polymers, and polyethylene glycol.

In some embodiments, the antibody formulation further comprises a surfactant. In some embodiments, the surfactant is selected from the group consisting of polysorbate, pluronics, Brij, and other nonionic surfactants. In some embodiments, the surfactant is polysorbate 80. The surfactant concentration in the formulation can vary. For example, in some embodiments the surfactant concentration in the formulation is about 0.001% to about 1%, about 0.005% to about 0.5%, about 0.0.01% to about 0.1%, or about 0.05% to about 0.07%.

In some embodiments, the antibody formulation further comprises histidine. In some embodiments, the histidine concentration in the formulation is about 5 mM to about 200 mM, about 10 mM to about 100 mM, about 20 mM to about 50 mM, or about 25 mM.

In some embodiments, various components can be omitted from the antibody formulation, or can be “substantially free” of that component. The term “substantially free” as used herein refers to an antibody formulation, said formulation containing less than 0.01%, less than 0.001%, less than 0.0005%, less than 0.0003%, or less than 0.0001% of the designated component.

In some embodiments, the formulation is substantially free of trehalose, i.e., the antibody formulation contains less than 0.01%, less than 0.001%, less than % 0.0005%, less than 0.0003%, or less than 0.0001% of trehalose. In some embodiments, the formulation comprises trehalose in a concentration of about 10 mM to about 1000 mM, about 50 mM to about 500 mM, about 100 mM to about 350 mM, about 150 mM to about 250 mM, about 180 mM or about 225 mM. In some embodiments, trehalose is used in combination with arginine. The concentrations of arginine and trehalose can vary and can be independent of each other. In some embodiments, the molar ratio of arginine:trehalose can be about 0:1, about 1:20, about 1:10, about 1:8, about 1:5, about 1:2, about 1:1, about 2:1, about 5:1, about 10:1, or about 10:0.

In some embodiments, the antibody formulation is substantially free of a saccharide, i.e., the antibody formulation, said formulation containing less than 0.01%, less than 0.001%, less than 0.0005%, less than 0.0003%, or less than 0.0001% of a saccharide. The term “saccharide” as used herein refers to a class of molecules that are derivatives of polyhydric alcohols. Saccharides are commonly referred to as carbohydrates and may contain different amounts of sugar (saccharide) units, e.g., monosaccharides, disaccharides and polysaccharides. In some embodiments, the formulation is substantially free of disaccharide. In some embodiments, the formulation substantially free of a reducing sugar, a non-reducing sugar, or a sugar alcohol. In some embodiments, the antibody formulation is substantially free to histidine, proline, glutamate, sorbitol, divalent metal ions, and/or succinate.

In some embodiments, the invention is directed to a stable, low viscosity antibody formulation comprising: (a) about 150 mg/mL to about 400 mg/mL of an antibody, e.g., an anti-IL6 antibody, (b) 150 mM to 400 mM arginine, (c) 0.01% to 0.1% polysorbate 80, (d) 5 mM to 100 mM histidine, wherein the antibody formulation has a viscosity of less than 20 cP at 23° C. In some embodiments, the antibody formulation comprises (a) 150 mg/mL of an antibody, e.g., an anti-IL6 antibody, (b) 25 mM histidine (e.g., L-histidine/L-histidine hydrochloride monohydrate), (c) 220 mM arginine (e.g., arginine HCl), and (d) 0.07% (w/v) polysorbate 80, at a pH 6.0. In some embodiments, the antibody formulation comprises (a) 150 mg/mL of an antibody, e.g., an anti-IL6 antibody, (b) 25 mM histidine (e.g., L-histidine/L-histidine hydrochloride monohydrate), (c) 150 mM arginine (e.g., arginine HCl), and (d) 0.07% (w/v) polysorbate 80, at a pH 6.0.

In some embodiments, the invention is directed to a stable, low viscosity antibody formulation comprising: (a) about 50 mg/mL to about 200 mg/mL of an antibody, e.g., an anti-IL6 antibody, (b) 20 mM to 400 mM arginine, (c) 0.01% to 0.1% polysorbate 80, (d) 5 mM to 100 mM histidine, and optionally (e) about 50 mM to about 400 mM trehalose, wherein the antibody formulation has a viscosity of less than 20 cP at 23° C. In some embodiments, the antibody formulation comprises (a) 50 mg/mL of an antibody, e.g., an anti-IL6 antibody, (b) 25 mM histidine (e.g., L-histidine/L-histidine hydrochloride monohydrate), (c) 225 mM trehalose, and (d) 0.05% (w/v) polysorbate 80, at a pH 6.0. In some embodiments, the antibody formulation comprises (a) 100 mg/mL of an antibody, e.g., an anti-IL6 antibody, (b) 25 mM histidine (e.g., L-histidine/L-histidine hydrochloride monohydrate), (c) 180 mM trehalose, (d) 25 mM arginine, and (e) 0.07% (w/v) polysorbate 80, at a pH 6.0.

In some embodiments, the invention is directed to a stable, low viscosity antibody formulation comprising: (a) about 150 mg/mL to about 400 mg/mL of an antibody, wherein the antibody comprises amino acid sequences of SEQ ID NOS:1 and 2, (b) 150 mM to 400 mM arginine, (c) 0.01% to 0.1% polysorbate 80, (d) 10 mM to 50 mM histidine, wherein the antibody formulation has a viscosity of less than 20 cP at 23° C. In some embodiments, the antibody formulation comprises (a) 150 mg/mL of an antibody, wherein the antibody comprises amino acid sequences of SEQ ID NOS:1 and 2, (b) 25 mM histidine (e.g., L-histidine/L-histidine hydrochloride monohydrate), (c) 220 mM arginine (e.g., arginine HCl), and (d) 0.07% (w/v) polysorbate 80, at a pH 6.0. In some embodiments, the antibody formulation comprises (a) 150 mg/mL of an antibody, wherein the antibody comprises amino acid sequences of SEQ ID NOS:1 and 2, (b) 25 mM histidine (e.g., L-histidine/L-histidine hydrochloride monohydrate), (c) 150 mM arginine (e.g., arginine HCl), and (d) 0.07% (w/v) polysorbate 80, at a pH 6.0.

In some embodiments, the invention is directed to a stable, low viscosity antibody formulation comprising: (a) about 50 mg/mL to about 200 mg/mL of an antibody, wherein the antibody comprises amino acid sequences of SEQ ID NOS:1 and 2, (b) 20 mM to 400 mM arginine, (c) 0.01% to 0.1% polysorbate 80, (d) 5 mM to 100 mM histidine, and optionally (e) about 50 mM to about 400 mM trehalose, wherein the antibody formulation has a viscosity of less than 20 cP at 23° C. In some embodiments, the antibody formulation comprises (a) 50 mg/mL of an antibody, wherein the antibody comprises amino acid sequences of SEQ ID NOS:1 and 2, (b) 25 mM histidine (e.g., L-histidine/L-histidine hydrochloride monohydrate), (c) 225 mM trehalose, and (d) 0.05% (w/v) polysorbate 80, at a pH 6.0. In some embodiments, the antibody formulation comprises (a) 100 mg/mL of an antibody, wherein the antibody comprises amino acid sequences of SEQ ID NOS:1 and 2, (b) 25 mM histidine (e.g., L-histidine/L-histidine hydrochloride monohydrate), (c) 180 mM trehalose, (d) 25 mM arginine, and (e) 0.07% (w/v) polysorbate 80, at a pH 6.0.

In some embodiments, the invention is directed to a method of treating a patient with an inflammatory pain component by administering the antibody formulation described herein. In some embodiments, the invention is directed to a method of treating a patient with an activated IL-6 dependent pathway by administering the antibody formulation described herein. In some embodiments, the invention is directed to a method of treating pain in a subject, the method comprising administering the antibody formulations described herein. In some embodiments, the invention is directed to a method of treating pain associated with osteoarthritis in a subject, the method comprising administering the antibody formulations described herein. In some embodiments, the invention is directed to a method of treating pain associated with chronic lower back pain in a subject, the method comprising administering the antibody formulations described herein.

As used herein, “subject” can be used interchangeably with “patient” and refers to any animal classified as a mammal, including humans and non-humans, such as, but not limited to, domestic and farm animals, zoo animals, sports animals, and pets. In some embodiments, subject refers to a human.

The terms “treat” and “treatment” refer to both therapeutic treatment and prophylactic, maintenance, or preventative measures, wherein the object is to prevent or alleviate (lessen) an undesired physiological condition, disorder or disease, or obtain beneficial or desired clinical results. The terms “treat,” “treatment,” and “treating” refer to the reduction or amelioration of the progression, severity, and/or duration of such a disease or disorder (e.g., a disease or disorder characterized by aberrant expression and/or activity of an IL-6 polypeptide, a disease or disorder characterized by aberrant expression and/or activity of an IL-6 receptor or one or more subunits thereof, an autoimmune disease, an inflammatory disease, a proliferative disease, or an infection (preferably, a respiratory infection)) or the amelioration of one or more symptoms thereof resulting from the administration of one or more therapies (including, but not limited to, the administration of one or more prophylactic or therapeutic agents). In certain embodiments, such terms refer to reduction in the pain associated with a various conditions. In other embodiments, such terms refer to the reduction of the release of inflammatory agents by mast cells, or the reduction of the biological effect of such inflammatory agents. In other embodiments, such terms refer to a reduction of the growth, formation and/or increase in the number of hyperproliferative cells (e.g., cancerous cells). In yet other embodiments, such terms refer to the eradication, removal or control of primary, regional or metastatic cancer (e.g., the minimization or delay of the spread of cancer). In yet other embodiments, such terms refer to the eradication, removal or control of (e.g., the minimization or delay of the spread of cancer) of non-small cell lung cancer. In yet other embodiments, such terms refer to the eradication, removal or control of rheumatoid arthritis. In some embodiments, the invention is directed to a method of treating rheumatoid arthritis in a subject, the method comprising administering the antibody formulations described herein.

In some embodiments, a therapeutically effective amount of the antibody formulations described herein is administered to treat a condition. As used herein, the term “therapeutically effective amount” refers to the amount of a therapy (e.g., an antibody that immunospecifically binds to an IL-6 polypeptide), that is sufficient to reduce the severity of a disease or disorder (e.g., a disease or disorder characterized by aberrant expression and/or activity of an IL-6 polypeptide, a disease or disorder characterized by aberrant expression and/or activity of an IL-6 receptor or one or more subunits thereof, an autoimmune disease, an inflammatory disease, a proliferative disease, or an infection (preferably, a respiratory infection) or one or more symptoms thereof), reduce the duration of a respiratory condition, ameliorate one or more symptoms of such a disease or disorder, prevent the advancement of such a disease or disorder, cause regression of such a disease or disorder, or enhance or improve the therapeutic effect(s) of another therapy. In some embodiments, the therapeutically effective amount cannot be specified in advance and can be determined by a caregiver, for example, by a physician or other healthcare provider, using various means, for example, dose titration. Appropriate therapeutically effective amounts can also be determined by routine experimentation using, for example, animal models.

The terms “therapies” and “therapy” can refer to any protocol(s), method(s), and/or agent(s) that can be used in the prevention, treatment, management, or amelioration of a disease or disorder (e.g., a disease or disorder characterized by aberrant expression and/or activity of an IL-6 polypeptide, a disease or disorder characterized by aberrant expression and/or activity of an IL-6 receptor or one or more subunits thereof, an autoimmune disease, an inflammatory disease, a proliferative disease, or an infection (preferably, a respiratory infection) or one or more symptoms thereof). In certain embodiments, the terms “therapy” and “therapy” refer to anti-viral therapy, anti-bacterial therapy, anti-fungal therapy, biological therapy, supportive therapy, and/or other therapies useful in treatment, management, prevention, or amelioration of such a disease or disorder or one or more symptoms known to skilled medical personnel.

As used herein, the term “therapeutic protocol” refers to a regimen for dosing and timing the administration of one or more therapies (e.g., therapeutic agents) that has a therapeutic effective.

The route of administration of the antibody formulation of the present invention can be via, for example, oral, parenteral, inhalation or topical modes of administration. The term parenteral as used herein includes, e.g., intravenous, intraarterial, intraperitoneal, intramuscular, subcutaneous, rectal or vaginal administration. In some embodiments, the isolated antibody is an anti-IL6 antibody (e.g., anti-IL6(YTE) antibody) and the route of administration is subcutaneous injection. While all these forms of administration are clearly contemplated as being within the scope of the invention, in some embodiments, the antibody formulation is suitable for administration via injection, in particular for intravenous or intraarterial injection or drip.

In some embodiments, the antibody formulation is diluted into an intravenous formulation prior to administration to a subject. In some instances, visible particle formation can occur upon dilution of the antibody formulation into the intravenous formulation, e.g., an IV bag. In order to address particle formation, in some embodiments, a method is provided to reduce the formation of particles when diluting an antibody formulation into an intravenous bag, the method comprising adding a buffer and a surfactant to the intravenous bag prior to adding the antibody formulation.

The term “IV bag protectant” refers to the surfactant added to the intravenous bag prior to dilution of the antibody formulation described herein into the intravenous bag. The IV bag protectant can also be added to the intravenous bag prior to addition of other antibody formulations known to those of skill in the art, e.g., a lyophilized antibody formulation.

Surfactants suitable for use as an IV bag protectant will generally be those suitable for use in IV formulations. In some embodiments, the surfactant used in the IV bag protectant is the same buffer used in the antibody formulation. For example, if the antibody formulation comprises polysorbate 80 as a surfactant, then polysorbate 80 would be added to the intravenous bag prior to adding the antibody formulation to the intravenous bag.

In some embodiments, the IV bag protectant comprises a surfactant which, when added to an IV formulation, will produce a surfactant concentration in the range of about 0.006% to about 0.018% surfactant, about 0.008% to about 0.015% surfactant, about 0.009% to about 0.012% surfactant, about 0.009% surfactant, about 0.010% surfactant, about 0.011% surfactant or about 0.012% surfactant in the IV formulation. In some embodiments, the surfactant is polysorbate 80 (PS80) which, when added to an IV formulation, will produce a surfactant concentration in the range of about 0.006% to about 0.018% surfactant, about 0.008% to about 0.015% surfactant, about 0.009% to about 0.012% surfactant, about 0.009% surfactant, about 0.010% surfactant, about 0.011% surfactant or about 0.012% surfactant in the IV formulation. In some embodiments, the surfactant concentration in the IV bag resulting from addition of the IV protectant will be about the same, about half, or about one seventh of the surfactant concentration in the antibody formulation.

Knowing the desired final concentration of surfactant in the IV bag, one can formulate the desired concentration of the surfactant in the IV bag protectant. For example, in some embodiments, the IV bag protectant can comprise about 0.01% to about 10.0% surfactant, about 0.05% to about 5% surfactant, about 0.1% to about 2% surfactant, or about 0.5% to about 1% surfactant.

In some embodiments, the invention can be directed to a kit, the kit comprising (1) an antibody formulation, and (2) an IV protectant formulation. In some embodiments, the invention can be directed to a kit, the kit comprising (1) an antibody formulation, and (2) an IV protectant, the IV protectant comprising a surfactant. In some embodiments, the surfactant is polysorbate 80. In some embodiments, the invention can be directed to a kit, the kit comprising (1) an antibody formulation as described herein, and (2) an IV protectant. In some embodiments, the invention can be directed to a kit, the kit comprising (1) an antibody formulation as described herein, and (2) an IV protectant, wherein (i) the IV protectant comprises polysorbate 80 in an amount sufficient to produce polysorbate 80 in the range of about 0.006% to about 0.018% when added to an IV formulation.

In some embodiments, the invention is directed to a method of pretreating an IV formulation, e.g., an IV bag, prior to dilution of an antibody formulation into the IV formulation, the method comprising (1) adding an IV protectant as described herein in the IV formulation, and (2) adding the antibody formulation.

In some embodiments, the invention is directed to a method of making a stable, low viscosity antibody formulation, the method comprising: (a) concentrating an antibody to about 150 mg/mL to about 400 mg/mL; and (b) adding arginine to the antibody of (a) to achieve an antibody formulation having a concentration of arginine of greater than about 150 mM. In some embodiments, the method further comprises (c) adding histidine to achieve an antibody formulation having a concentration of histidine of 10 mM to 100 mM. In some embodiments, the method further comprises (d) adding a surfactant, e.g., polysorbate 80, to achieve an antibody formulation having a concentration of surfactant of 0.02% to 0.1%.

In some embodiments, the invention is directed to a method of making a stable, low viscosity antibody formulation, the method comprising: (a) concentrating an antibody to about 100 mg/mL to about 400 mg/mL; and (b) adding arginine to the antibody of (a) to achieve an antibody formulation having a concentration of arginine of about 100 mM to about 200 mM. In some embodiments, the method further comprises (c) adding histidine to achieve an antibody formulation having a concentration of histidine of 10 mM to 100 mM. In some embodiments, the method further comprises (d) adding a surfactant, e.g., polysorbate 80, to achieve an antibody formulation having a concentration of surfactant of 0.02% to 0.1%. In some embodiment, the method further comprises adding trehalose to achieve an antibody formation having a concentration of trehalose of about 100 mM to about 300 mM.

In some embodiments, the invention is directed to a method of making a stable, low viscosity antibody formulation, the method comprising: (a) concentrating an antibody to about 50 mg/mL to about 400 mg/mL; and (b) adding trehalose to the antibody of (a) to achieve an antibody formulation having a concentration of trehalose of about 100 mM to about 400 mM. In some embodiments, the method further comprises (c) adding histidine to achieve an antibody formulation having a concentration of histidine of 10 mM to 100 mM. In some embodiments, the method further comprises (d) adding a surfactant, e.g., polysorbate 80, to achieve an antibody formulation having a concentration of surfactant of 0.02% to 0.1%.

In some embodiments, the invention is directed to a method of making a stable, low viscosity antibody formulation, the method comprising: (a) concentrating an antibody to about 150 mg/mL to about 400 mg/mL, wherein the antibody comprises amino acid sequences of SEQ ID NOS:1 and 2; and (b) adding arginine to the antibody of (a) to achieve an antibody formulation having a concentration of arginine of greater than about 150 mM, wherein the antibody formulation of (b) is in an aqueous solution and has a viscosity of less than 20 cP at 23° C., and wherein the antibody formulation of (b) is stable at 2° C. to 8° C. for 12 months as determined by SEC HPLC.

In some embodiments, the compositions and methods of the present invention enable a manufacturer to produce an antibody formulation suitable for administration to a human in a more efficient manner, either by reducing costs, reducing method steps, reducing opportunities for error, reducing opportunities for introduction of unsafe or improper additives, etc. In the present invention, antibody formulations can be administered without reconstitution of lyophilized antibody.

### EXAMPLES

### Example 1

### Materials and Methods

### Materials

All the materials used were of USP or Multicompendial grade. All the solutions and buffers were prepared using USP or HPLC water and were filtered through 0.2 μm PVDF filters (Millipore, Millex GV, SLG033RB) before further use. Purified anti-IL6(YTE) was purified. Purified anti-IL6(YTE) samples for stability studies were prepared under sterile aseptic conditions in the Biosafety Cabinet Hood (BSC). Bulk material was stored at 2-8° C.

### Methods

i. Protein Concentration Determination

Anti-IL6(YTE) antibody concentrations were determined by measuring absorbance at 280 nm with an Agilent UV-Vis spectrophotometer. A measured extinction coefficient of 1.71 (mg/mL)⁻¹ cm⁻¹ was used to calculate protein concentrations.

ii. Purity Determination by Size Exclusion Chromatography

Size Exclusion Chromatography (SEC) analysis was performed on an Agilent HPLC system with a TSK-GEL G3000SWXL column and SW guard column (Tosh Bioscience LLC, Mongomeryville, Pa.) with UV detection at 280 nm. A flow rate of 1.0 mL/min for 20 minutes using a pH 6.8 mobile phase containing 0.1 M sodium phosphate, 0.1 M sodium sulfate, and 0.05% (w/v) sodium azide was used to assay the samples. About 250 micrograms of protein was injected. Elution of soluble aggregates, monomer, and fragments occurred at approximately 6 to 8 minutes, 8.5 minutes, and 9 to 11.5 minutes respectively.

iii. Determination of Fragmentation Level by Reversed Phase Chromatography

Fragmentation levels were measured using an Agilent HPLC system with a Michrom Bioresources PLRP-S CM810092/00 column.

iv. Visual Appearance

Visual inspection was performed for visible particles, clarity/opalescence, and color following procedures adapted from the PhEur (sections 2.9.20, 2.2.1 and 2.2.2 respectively).

v. Sub-Visible Particle Analysis

Sub-visible particles analysis was performed using either light obscuration (HIAC 9705) or Flow microscopy (Brightwell Microflow Imager, MFI).

vi. Osmolality

Osmolality was measured using Advance Instrument Inc. 2020 freezing point depression osmometer.

vii. Viscosity Assessment

The viscosities of anti-IL6(YTE) formulations at various concentrations were measured using an Anton Paar MCR301 Rheometer.

viii. Formulation Stability Studies

Anti-IL6(YTE) antibody formulated with different excipients was filled into clear 3 cc, 13 mm glass vials. For accelerated screening, samples were placed on stability at 40° C./75% RH and at 25° C./60% RH and 5° C. Samples were analyzed by SEC HPLC, RP HPLC, and the vials were visually inspected for particles. In addition selected time points were analyzed for potency, osmolality, pH, HIAC, and MFI as appropriate.

ix. Colloidal Stability Screening using Turbidity

Colloidal stability was screened by measuring the turbidity of various anti-IL6 antibody formulations vs. time using a Cary Eclipse multicell UV-Vis spectrophotometer when subjected to elevated temperature of about 62° C. Less stable formulations become turbid as they form particulates and precipitates (i.e. have a higher absorbance at 360 nm) over time whereas more colloidally stable formulations remain clear for a longer duration.

x. Thermal Stability using Differential Scanning calorimetry

Differential scanning calorimetry (DSC) experiments were performed on a VP-DSC Ultrasensitive Differential scanning calorimeter (Microcal, Northampton, Mass.) using 96 well plate at a protein concentration of 1 mg/mL. Samples were heated from 20-100° C. at a rate of 90° C. per hour. Normalized heat capacity (Cp) data were corrected for buffer baseline. The first melting transition (Tₘ₁) and the second melting transition (Tₘ₂) were used to rank order excipients according to their stabilizing effect on the conformational stability of the protein.

xi. Thermal Stability Using Differential Scanning Fluorimetry

Differential Scanning Fluorometry (DSF) experiments were performed at a protein concentration of about 0.5 mg/mL with SYPRO orange dye (Invitrogen, S6651) at a 5× level (the original concentration is 5000×). Stocks of excipients were mixed with protein/dye stock (ca. 5 mg/mL protein and 50× dye) in a ratio of 9:1 to achieve the target levels formulated in isotonic solutions of various excipients. The dye along with the protein solution and the buffer/excipient was mixed thoroughly for 25 μl per well in a 96 well plate. Fluorescence increases due to dye-binding to unfolded protein molecules was measured using a BioRad C1000 Thermal Cycler PCR plate reader. Samples were run in triplicate and were heated from 20-90° C. in 0.2° C. increments for 10 s per reading resulting in a rate of 1.2° C./min. The inflection point in the fluorescence was reported as Th, a measure of the conformational stability of the protein.

### Example 2

### Conformational Thermal Stability

The effect that various excipients have on conformational (thermal) stability of anti-IL6(YTE) antibody was investigated as described in Example 1. The results are presented in Table 1.

TABLE 1
Conformational (Thermal) stability: ranked excipient effects

| Excipient | DSF | DSC | DSC |
|---|---|---|---|
| (approx mM level) | (Th) | (Tm1) | (Tm2) |
| 300 mM trehalose | 61.1 | 64.9 | 71.7 |
| 300 mM glycine | 59.5 | 63.9 | 71.7 |
| 25 mM histidine pH 6 control | 59.6 | 63.4 | 70.4 |
| 167 mM phosphate | 59.6 | Not done | Not done |
| 25 mM phosphate pH 6 | 59.5 | Not done | Not done |
| 300 mM sucrose | 59.5 | 63.2 | 71.8 |
| 300 mM mannitol | 59.5 | Not done | Not done |
| 150 mM glutamate | 59.4 | Not done | Not done |
| 25 mM citrate pH 6 | 59.4 | Not done | Not done |
| 150 mM NaOAc | 59.3 | Not done | Not done |
| 115 mM citrate | 59.2 | Not done | Not done |
| 150 mM aspartate | 59.2 | Not done | Not done |
| 150 mM NaCl | 59.0 | 61.4 | 69.7 |
| 143 mM succinate | 59.0 | Not done | Not done |
| 231 mM histidine | 58.7 | Not done | Not done |
| 150 mM NaSulfate | 58.0 | Not done | Not done |
| 150 mM lysine | 57.5 | Not done | Not done |
| 150 mM arginine | 57.1 | 60.6 | 70.2 |
| 220 mM arginine | Not done | 59.9 | 70.0 |

As can be seen in Table 1, arginine was the least conformationally stabilizing excipient, especially when compared to the base buffer conditions of 25 mM histidine.

Further investigation demonstrated that arginine wasn't even predicted to be the most colloidally stabilizing excipient for anti-IL6(YTE) antibody as can be seen in FIG. 1. The most colloidally stabilizing excipients were sucrose and trehalose while the least stabilizing were NaCl and sodium sulfate.

### Example 3

### Viscosity and Stability Screening Assessments

The viscosity profiles, and stability, of multiple anti-IL6(YTE) antibody formulations were assessed as described in Example 1 and found to be to be acceptable from both stability and a predicted syringe functionality perspective. A viscosity of 14 cP was expected to result in acceptable syringe gliding force performance using thin-wall 27 gauge needles for prefilled syringe products (ca. 7 N injection force and 9-16 s injection time).

Table 2 summarizes an investigation into the impact of pH, buffer type, histidine level, and arginine level on the stability and viscosity of anti-IL6(YTE) formulations at 100 mg/mL.

TABLE 2

| | | | | | | | SEC Purity |
|---|---|---|---|---|---|---|---|
| | | Arginine | | Trehalose | Polysorbate | Viscosity | Loss Rate |
| Sample # | Buffer | (mM) | pH | (mM) | (% w/v) | (cP) | at 40° C. |
| 1 pH | 25 mM | 50 | 5.0 | 225 | 0.05 | 8.3 | 3.2 |
| 5.0 | Acetate | | | | | | |
| 2 pH | 25 mM | 50 | 5.5 | 225 | 0.05 | 7.5 | 2.3 |
| 5.5 | Succinate | | | | | | |
| 3 | 25 mM | 50 | 6.0 | 225 | 0.05 | 6.8 | 2.1 |
| 50 mM arg | Histidine | | | | | least | most |
| | | | | | | viscous | stable |
| 4 | 25 mM | 25 | 6.0 | 225 | 0.05 | 8.1 | 2.6 |
| 25 mM arg | Histidine | | | | | | |
| 5 | 25 mM | 0 | 6.0 | 225 | 0.05 | 9.1 | 2.7 |
| Base Case | Histidine | | | | | | |
| 6 | 75 mM | 0 | 6.0 | 225 | 0.05 | 7.5 | 2.5 |
| higher buffer | Histidine | | | | | | |
| strength | | | | | | | |

Samples 1, 2, and 3 show that anti-IL6(YTE) antibody formulations are less stable and more viscous at lower pHs. Samples 5, 4, and 3, show that increasing the arginine levels in the anti-IL6(YTE) antibody formulations results in higher stability and lower viscosity, both desirable properties. Samples 5 and 6 show that increasing the histidine buffer strength can also reduce viscosity and increase stability. The approach of adding histidine was not pursued further because of the known potential issues with yellowing over time. These results show that the viscosity and stability was acceptable over the pH range of 5 to 6 with all combinations tested. Higher arginine levels at pH 6.0 seems optimal for both stability and viscosity of anti-IL6(YTE).

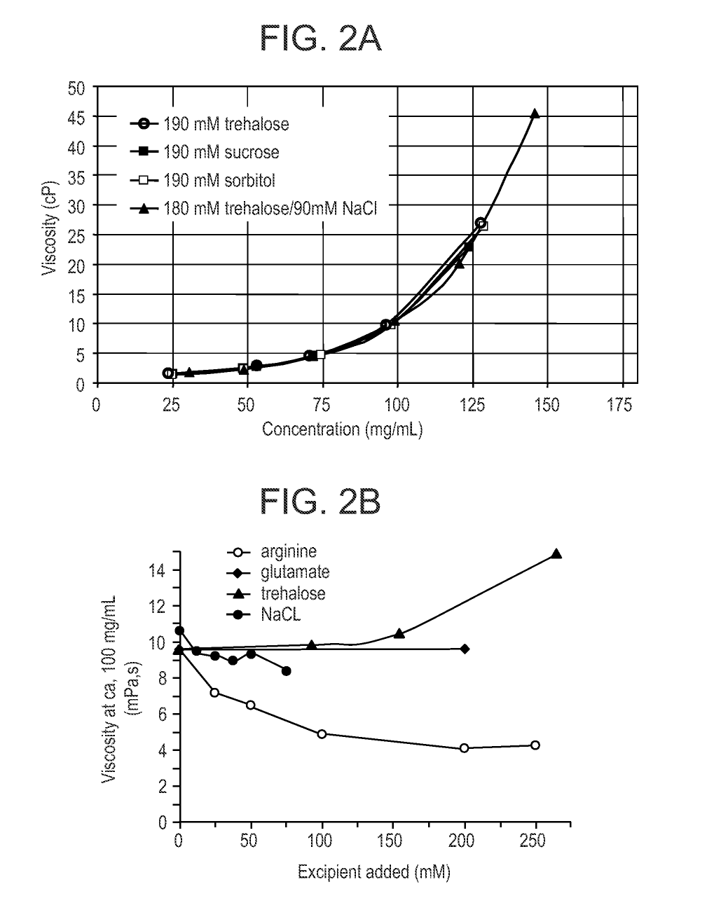

The viscosity profile of anti-IL6(YTE) antibody formulations using various excipients was assessed to determine what conditions would be optimal for a 150 mg/mL formulation. See FIG. 2A. Trehalose, sucrose and sorbitol had similar viscosity profiles to each other, and salt did not effectively reduce the viscosity. The data indicates that salts have an inability to reduce the viscosity of the antibody formulations. FIG. 2B demonstrates the effect that arginine, glutamate, sodium chloride, and trehalose have on viscosity.

The effect of various additional excipients on anti-IL6(YTE) antibody formulations viscosity was investigated. The results are found in Table 3.

TABLE 3

| | Vis- | Concen- |
|---|---|---|
| | cosity | tration |
| Formulation | ((cP) | (mg/mL) |
| 10% Trehalose, 25 mM histidine, pH 6.0 | 14.9 | 102 |
| 10% Sucrose, 10 mM NaCl, 25 mM histidine, pH 6.0 | 11.8 | 108 |
| 10% Trehalose, 10 mM CaCl₂, | 11.8 | 109 |
| 25 mM histidine, pH 6.0 | | |
| 10% Trehalose, 10 mM NaCl, 25 mM histidine, | 11.7 | 104 |
| pH 6.0 | | |
| 10% Sucrose, 25 mM histidine, pH 6.0 | 10.8 | 102 |
| 10% Trehalose, 25 mM histidine, pH 5.5 | 10.8 | 102 |
| 6% Trehalose, 50 mM NaCl, 25 mM histidine, pH 6.0 | 8.5 | 100 |
| 6% Trehalose, 50 mM Lysine, 25 mM histidine, | 8.5 | 106 |
| pH 6.0 | | |
| 6% Sucrose, 50 mM Lysine, 25 mM histidine, pH 6.0 | 8.3 | 106 |
| 6% Sucrose, 50 mM Arginine, 25 mM histidine, | 7.9 | 107 |
| pH 6.0 | | |
| 25 mM histidine, pH 6.0 | 7.6 | 93 |
| 50 mM NaCl, 25 mM histidine, pH 6.0 | 7.6 | 98 |
| 6% Trehalose, 50 mM Arginine, 25 mM histidine, | 7.3 | 107 |
| pH 6.0 | | |
| 150 mM NaCl, 25 mM histidine, pH 6.0 | 6.7 | 101 |

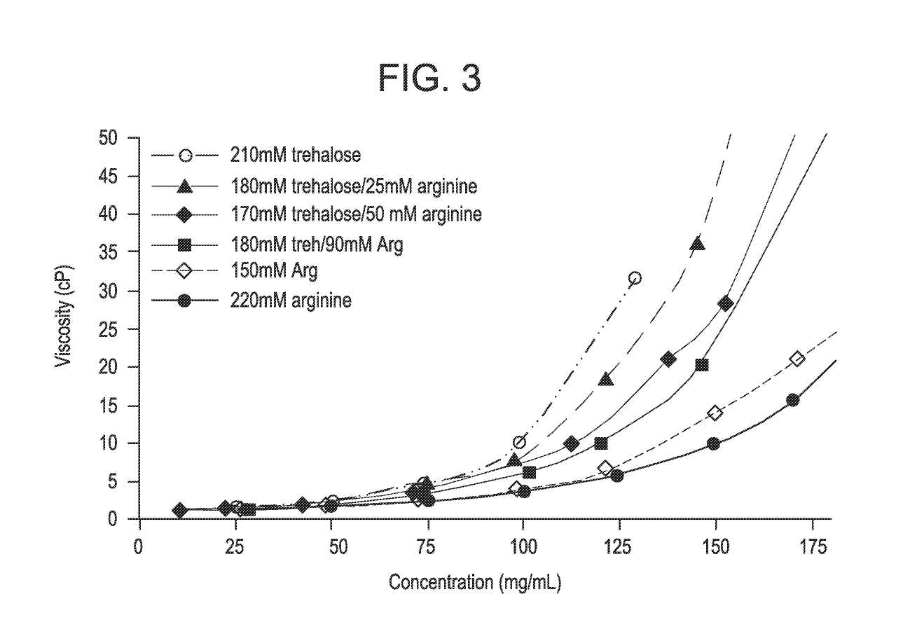

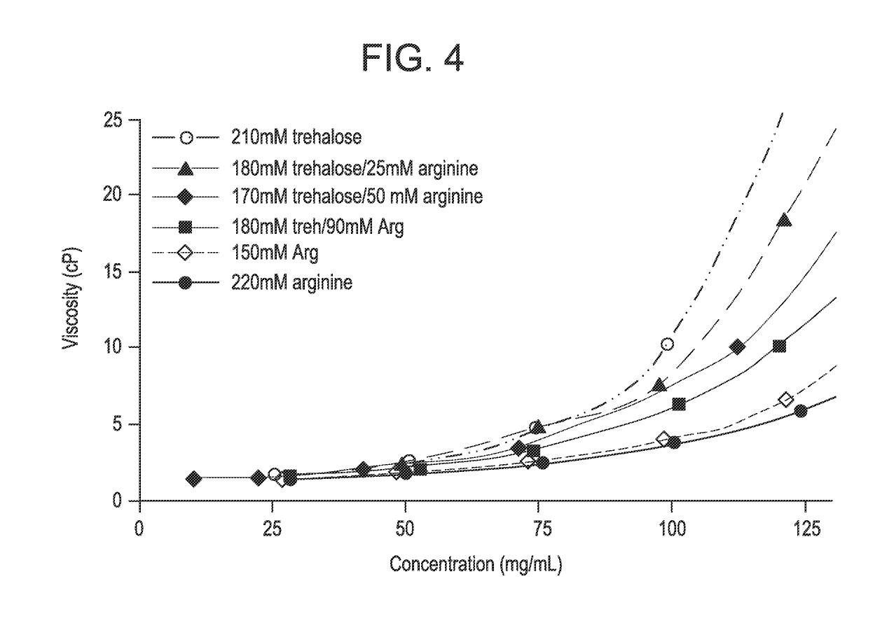

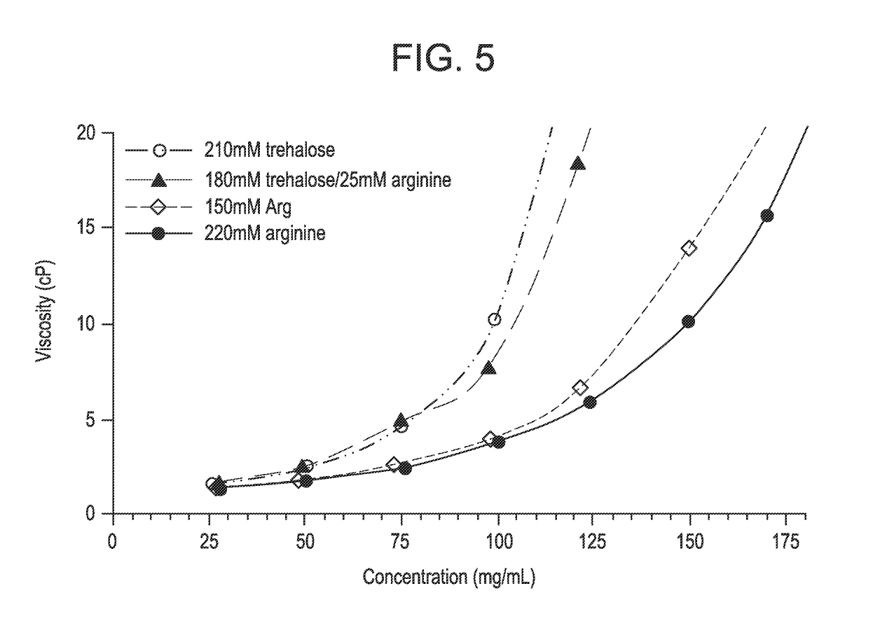

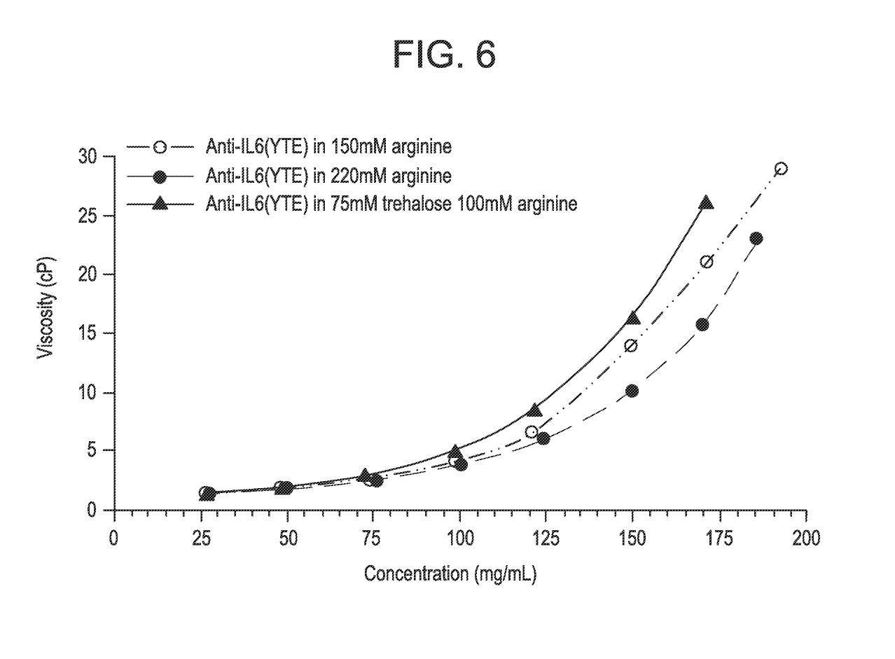

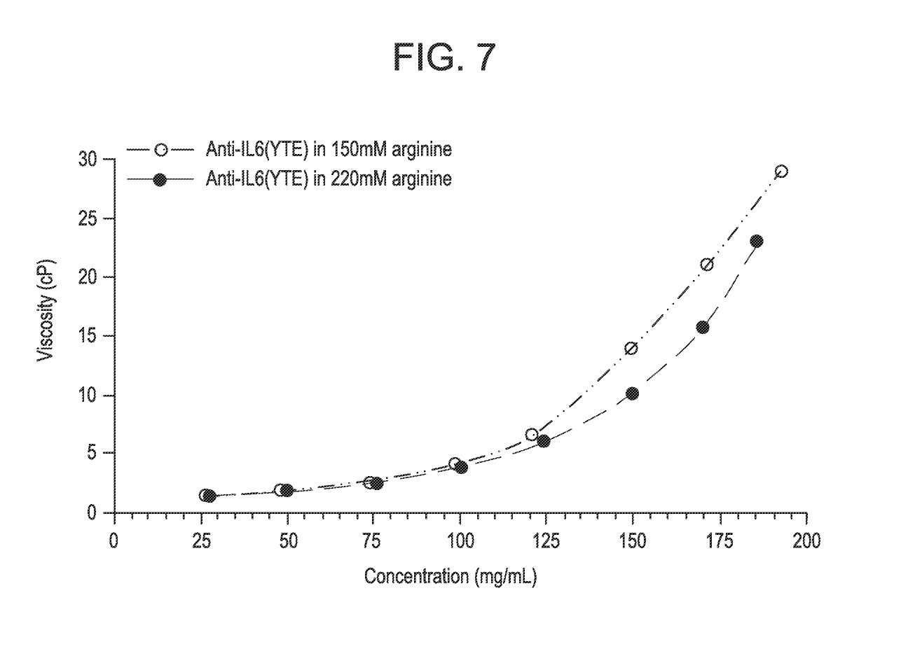

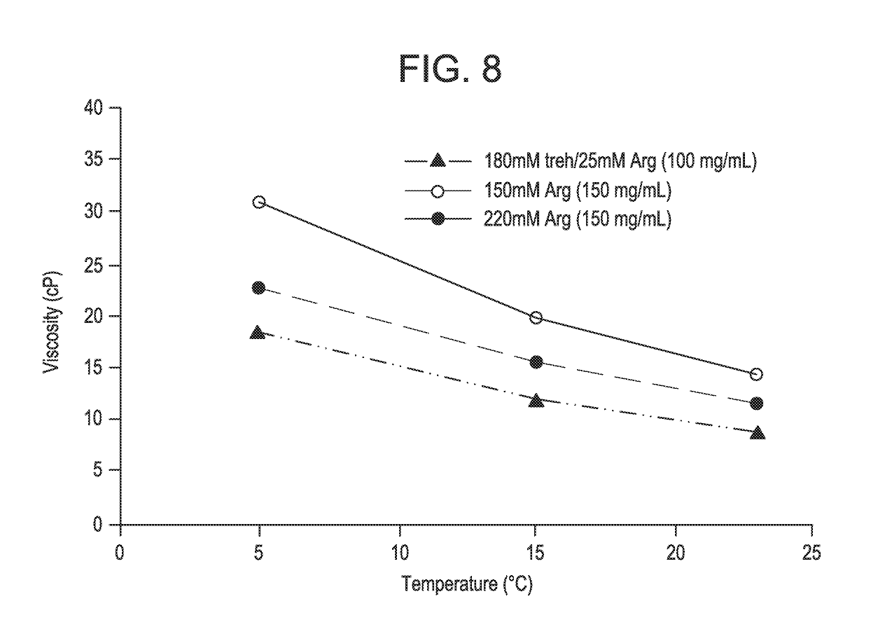

Increased arginine levels resulted in lower viscosity profiles (FIG. 3 and FIG. 4). As low as 25 mM arginine is able to reduce the viscosity to below 10 cP nominal at 100 mg/mL. To achieve a 150 mg/mL antibody formulation, 150 mM arginine and 220 mM arginine are both able to reduce the viscosity to below about 15 cP nominal, with the higher 220 mM arginine option being substantially lower at about 10 cP (FIG. 5). The data suggests that 150 mM arginine is necessary to meet a target of <20 cP as shown in the attempt to try 100 mM arginine with 75 mM trehalose (FIG. 6). The 220 mM arginine anti-IL6(YTE) formulation has lower viscosity profile than the 150 mM arginine by about 5 cP at ca. 185 mg/mL (the over-concentration level), see FIG. 7. FIG. 8 shows the temperature dependence of the viscosities for the leading 100 and 150 mg/mL formulations.

### Example 4

### Study of Impact of Excipient on Stability and Viscosity

Experiments to assess the impact of trehalose and arginine on multiple formulation parameters were performed. The antibody formulation was stored at either 40° C. or 5° C., and the purity loss was determined at various times. High performance Size Exclusion Chromatography was performed as described in Example 1 using a TSK-GEL G3000SWXL column and SW guard column (Tosh Bioscience LLC, Mongomeryville, Pa.) with UV detection at 280 nm. The results are provided in Table 4.

TABLE 4

| | | | | | Purity loss | Purity Loss | Visual |
|---|---|---|---|---|---|---|---|
| Ab conc | | Viscosity | Osmo | Measured | Rate at 40° C. | Rate at 5° C. | appearance at |
| (mg/mL) | Formulation | (cP) | (mosm/kg) | Tm1 (° C.) | (%/month) | (%/yr) | 5° C. |
| 50 | 25 mM his | 3 | 321 | 63.8 | 3.7 | 0.6 | Pass |
| | 225 mM treh | | | | | | |
| | 0.05% PS80 | | | | | | |
| | pH 6.0 | | | | | | |
| 100 | 25 mM his | 9-13 | 311 | Not | 2.3 | 1.2 (9 mo) | Pass |
| | 180 mM treh | | | measured | | 1.1 (12 mo) | 9 months |
| | 25 mM arg | | | | | | Pass |
| | 0.07% PS80 | | | | | | 12 months |
| | pH 6.0 | | | | | | |
| 150 | 25 mM his | 14-19 | 325 | 60.6 | 1.2 | 1.2 (9 mo) | Pass |
| | 150 mM arg | | | | | 0.6 (12 mo) | 9 months |
| | 0.07% PS80 | | | | | | Pass |
| | pH 6.0 | | | | | | 12 months |
| 150 | 25 mM his | 10-14 | 448 | 59.9 | 1.4 | 0.8 (9 mo) | Pass |
| | 220 mM arg | | | | | 0.3 (12 mo) | 6 months |
| | 0.07% PS80 | | | | | | Pass |
| | pH 6.0 | | | | | | 12 months |

“Pass” indicated that the formulation was practically free from visible particles. These assessments demonstrate that anti-IL6(YTE) is stable at 100 mg/mL or above in the trehalose and arginine formulations provided above.

### Example 5

### Anti-IL6(YTE) Thermostability

An anti-IL6 antibody formulation was made containing anti-IL6 antibody at 150 mg/mL in 25 mM L-histidine/L-histidine hydrochloride monohydrate, 220 mM Arginine hydrochloride, 0.07% (w/v) polysorbate 80, pH 6.0. The composition of this formulation is outlined in Table 5.

TABLE 5

| | | | | |
|---|---|---|---|---|
| | Unit | | | |
| | Formula | | | |
| | per 150 | | | |
| | mg Vial | | Quality | |
| Ingredient | (nominal) | Purpose | Standard | Concentration |

Active Ingredient

| | | | | | |
|---|---|---|---|---|---|
| Anti-IL6 | 150 mg | Active | In-house | 150 | mg/mL |
| antibody | | | Reference | | |
| | | | Standard | | |

Excipients

| | | | | | |
|---|---|---|---|---|---|
| L-Histidine | 1.6 mg | Formulation | USP; EP | 10 | mM |
| | | buffer | | | |
| L-Histidine | 3.1 mg | Formulation | EP | 15 | mM |
| hydrochloride | | buffer | | | |
| monohydrate | | | | | |
| Arginine | 46.3 mg | Stabilizer, | USP; NF; | 220 | mM |
| hydrochloride | | tonicity | EP | | |
| | | modifier, | | | |
| | | viscosity | | | |
| | | modifier | | | |
| Polysorbate 80 | 0.7 mg | Adsorption | NF; EP | 0.07% | (w/v) |
| (plant derived) | | inhibitor | | | |

| | | | | |
|---|---|---|---|---|
| Water for | 855 | Aqueous | USP; EP | 47M |
| Injection | | vehicle | | |

EP = European Pharmacopoeia;
NA = not applicable;
NF = National Formulary;
USP = United States Pharmacopoeia

An anti-IL6 antibody formulation was made containing anti-IL6 antibody at 150 mg/mL in 25 mM L-histidine/L-histidine hydrochloride monohydrate, 150 mM Arginine hydrochloride, 0.07% (w/v) polysorbate 80, pH 6.0. The composition of this formulation is outlined in Table 6

TABLE 6

| | | | | |
|---|---|---|---|---|
| | Unit | | | |
| | formula | | | |
| | per 150 | | | |
| | mg Vial | | Quality | |
| Ingredient | (nominal) | Purpose | Standard | Concentration |

Active Ingredient

| | | | | | |
|---|---|---|---|---|---|
| Anti-IL6 | 150 mg | Active | In-house | 150 | mg/mL |
| antibody | | | Reference | | |
| | | | Standard | | |

Excipients

| | | | | | |
|---|---|---|---|---|---|
| L-Histidine | 1.7 mg | Formulation | USP; EP | 11 | mM |
| | | buffer | | | |
| L-Histidine | 2.9 mg | Formulation | EP | 14 | mM |
| hydrochloride | | buffer | | | |
| monohydrate | | | | | |
| Arginine | 31.6 mg | Stabilizer, | USP; NF; | 150 | mM |
| hydrochloride | | tonicity | EP | | |
| | | modifier, | | | |
| | | viscosity | | | |
| | | modifier | | | |
| Polysorbate 80 | 0.7 mg | Adsorption | NF; EP | 0.07% | (w/v) |
| (plant derived) | | inhibitor | | | |

| | | | | |
|---|---|---|---|---|
| Water for | 866 | Aqueous | USP; EP | 48M |
| Injection | | vehicle | | |

EP = European Pharmacopoeia;
NA = not applicable;
NF = National Formulary;
USP = United States Pharmacopoeia

The Drug Product was aseptically filled into 3 cc glass vials, stoppered and sealed with an aluminum overseal.

### Thermal Stability of the Anti-IL6(YTE) Antibody

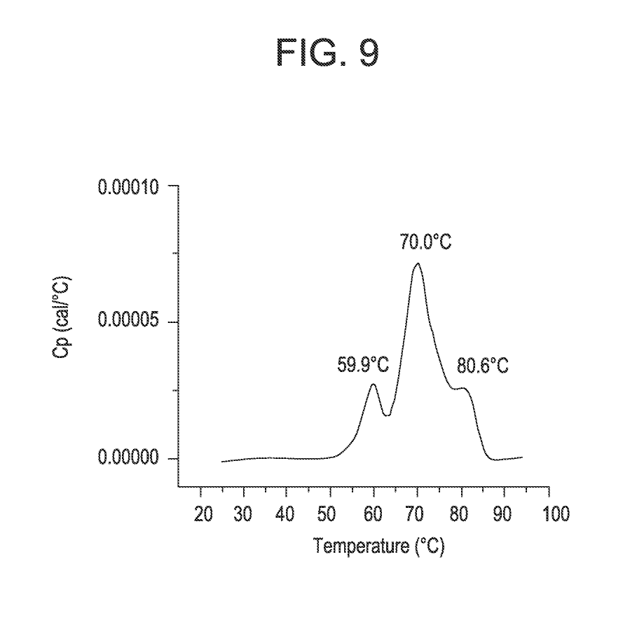

DSC was run on anti-IL6(YTE) at about 1 mg/mL in the formulation presented in Table 5 (25 mM L-histidine/L-histidine hydrochloride monohydrate, 220 mM Arginine hydrochloride, 0.07% (w/v) polysorbate 80, pH 6.0.) The thermal stability profile is given in FIG. 9.

### Example 6

### IV Bag Protectant

i. Materials

A lyophilized formulation was used to assess compatibility of anti-IL6(YTE) antibody in intravenous infusion (IV) bags and lines of various types from multiple vendors. The anti-IL6(YTE) antibody was in a lyophilized form, which when reconstituted, resulted in 50 mg/mL anti-IL6(YTE) antibody in 25 mM L-histidine/L-histidine hydrochloride monohydrate, 225 mM (8.5% [w/v]) trehalose dihydrate, 0.05% (w/v) polysorbate 80, pH 6.0.

ii. Methods

### (a) Compatibility Testing Procedure.

The in-use stability of anti-IL6(YTE) antibody CSP held and delivered using IV bags (or bottles), IV filter extension sets, and related contact materials of various types available in the clinic was assessed. The testing range was between 20 mg and 600 mg using 100 mL IV bags (0.2 mg/mL to 6 mg/mL). The calculated anti-IL6(YTE) antibody dose volume was added to the bags and gently mixed. IV bags were stored uncovered at both room temperature (RT, approximately 23° C.) and also under refrigerated conditions (2-8° C.) for 24 hours. After the appropriate incubation time, the CSP in the IV bags was collected by mock-infusion at 100 mL/hr by either pump or by gravity through an IV administration, filter, and extension set with needle. Particle formation/precipitation stability, and recovery of anti-IL6(YTE) antibody in the CSP was assessed by visual inspection, HPSEC and ultraviolet-visible (UV-Vis) absorbance.

### (b) Visual Inspection.

Visual inspection was performed directly on IV bags and also on material mock-infused into 3 cc glass drug vials for visible particles, clarity/opalescence, and color following procedures adapted from the PhEur (sections 2.9.20, 2.2.1 and 2.2.2 respectively). The starting anti-IL6(YTE) antibody formulation was slightly opalescent and colorless-to-slightly-yellow. After mock-infusion, the anti-IL6(YTE) antibody CSPs were clear and colorless-to-slightly-yellow for all CSP samples. However, if an IVBP was not used, increased particles levels were observed upon dilution of anti-IL6(YTE) antibody into IV bags. Use of the IVBP mitigated the particle formation in the CSP.

### (c) Purity and Soluble Aggregation.

High Performance Size Exclusion Chromatography (HPSEC) was performed using a TSK-GEL G3000SWXL column and SW guard column (Tosoh Bioscience LLC, Montgomeryville, Pa.) to assess purity and soluble aggregation of CSP samples.

### (d) Concentration and Recovery.

Protein recovery was assessed by ultraviolet-visible (UV-Vis) absorbance at 280 nm to assay protein concentration using an Agilent Model 8453 UV-Vis Spectrophotometer (Santa Clara Calif.). For doses below the quantization limit of the UV-Vis, HPSEC with fluorescence excitation at 280 nm and emission at 335 nm, was used to assay the protein using a linear peak area standard calibration curve.

iii. Results and Discussion

### (a) Particle Formation in Saline IV Bags

In initial testing without the use of the IVBP, visible particles were observed for anti-IL6(YTE) antibody in 100 mL saline IV bags and in the material collected into 3 cc glass vials after mock-infusion through a 0.2 micron in-line filter (FIG. 10). All other tests results were acceptable. Because visible particles are generally larger than 70 μm, these visible particles must have formed after the 0.22 micron in-line filter. In fact, it was observed that the samples collected in the 3 cc glass vials developed increased levels of particles over the course of the inversions and swirling agitation during the manual visual inspection process. We hypothesized that the formation of particles was due to the fact that insufficient surfactant is present in the solution. To investigate this, additional polysorbate was spiked into the IV bags.

### (c) Investigation of Impact of Surfactant Level on Particle Formation

The effect of the up to approximately 250-fold dilution of polysorbate was evaluated (100 mL/0.4 mL=250 fold dilution). The saline IV fluid was modified with addition of polysorbate 80 prior to dosing the anti-IL6(YTE) antibody into the IV bag. The added polysorbate 80 was varied from 0% to 0.018% w/v and the visual inspection performed (Table 7).

TABLE 7

| Polysorbate 80 | Visual inspection results of |
|---|---|
| Level in IV Bag % (w/v) | particles in saline bag 5 |
| 0.0002 | Not acceptable |
| 0.006 | Not acceptable |
| 0.009 | Practically free of visible |
| | particles |
| 0.010 | Practically free of visible |
| | particles |
| 0.011 | Practically free of visible |
| | particles |
| 0.012 | Practically free of visible |
| | particles |
| 0.015 | Practically free of visible |
| | particles |
| 0.018 | Practically free of visible |
| | particles |

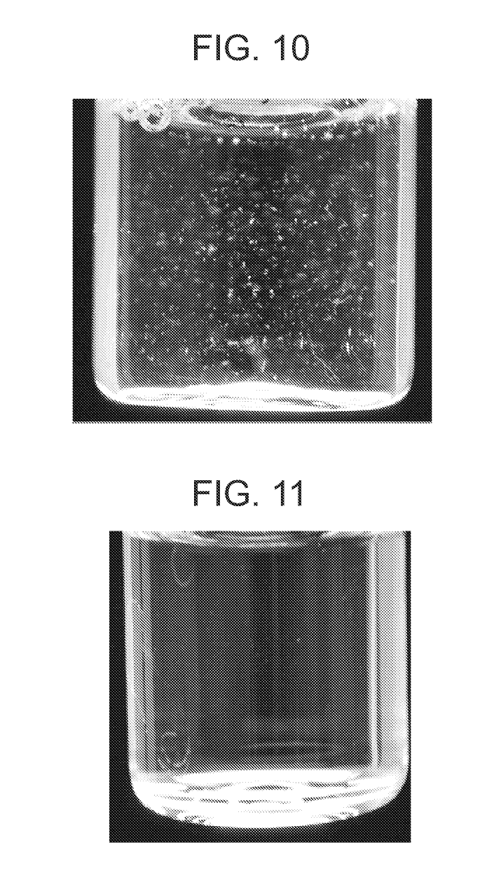

Note that for the 20 mg dose, a residual 0.0002% PS80 was contributed from the dilution of the polysorbate in the anti-IL6(YTE) antibody formulation volume added (0.05%/250=0.0002%). Based on these data, greater than 0.009% w/v of polysorbate 80 could effectively mitigate the observed particle formation in the CSP. FIG. 11 shows a photograph of anti-IL6(YTE) antibody in saline with 0.012% w/v of added polysorbate 80.

### (d) Use of an IV Bag Protectant (IVBP) to Mitigate Particle Formation in IV Bags

An IVBP was used to provide a higher level of polysorbate necessary to maintain stability of anti-IL6(YTE) antibody. A final level of 0.012% w/v polysorbate 80 was targeted for robustness in the level when accounting for errors and bags overfill variability. The IV bag protectant (IVBP) used was 0.65% (w/v) polysorbate 80 formulated in citrate buffer at pH 6.0. The IV bag preparation procedure was changed to call for the addition of a 1.8 mL volume of IVBP to be gently mixed before the anti-IL6(YTE) antibody dose was added. This resulted in a polysorbate level of about 0.012% w/v for the low doses and 0.018% w/v for the high doses. Compatibility studies were performed with the IVBP in five different saline IV bag types. These were found to be compatible with anti-IL6(YTE) antibody when the IVBP was used

iv. Conclusions

In this case study, the formation of proteinaceous particles in the CSP in the IV bags was the caused by the dilution of the polysorbate 80 below its protective level. It was determined that an IV bag protectant (IVBP) pre-treatment of the bag diluent was needed to keep the polysorbate level in the IV bag above the level necessary to mitigate particle formation (above about 0.009%) of the anti-IL6(YTE) antibody clinical sterile preparation (CSP). The IV bag protectant (IVBP) used was 0.65% (w/v) polysorbate 80 formulated in citrate buffer at pH 6.0 and was added to the bag before anti-IL6(YTE) antibody. Implementation of a polysorbate-containing IV bag protectant (IVBP) completely mitigated the particle formation for the anti-IL6(YTE) antibody CSP.

### Example 7

### Study of Impact of Excipient on Stability and Viscosity for Non-Anti-IL6 Antibody

Experiments to assess the impact of proline and arginine on multiple formulation parameters were performed. The anti-IL6 antibody and the non-anti-IL6 antibody (antibody X) formulation were stored at 40° C. and 5° C. and the purity loss and visible particle appearance was determined at various times. The thermal stability was determined using DSC (VP-DSC, Microcal, Northampton, Mass.). The viscosities of the formulations at were measured using an Anton Paar MCR301 Rheometer. High performance Size Exclusion Chromatography was performed as described in Example 1 using a TSK-GEL G3000SWXL column and SW guard column (Tosh Bioscience LLC, Mongomeryville, Pa.) with UV detection at 280 nm. The thermal stability was determined using DSC.

The results are provided in Table 8. Two antibody X formulations were compared. The two antibody X formulations were the same except that one had 50 mM arginine and the other had 50 mM proline. The results show that for antibody X that the visible appearance of particles in the arginine formulation was unacceptable after 11 weeks at 5° C. whereas the proline-containing formulation remained practically free of visible particles. Therefore, arginine had a negative impact on particle formation for the antibody X formulation. Both antibody X formulations had similar purity loss rates on stability indicating arginine did not either stabilize or destabilize, antibody X as measured by HP-SEC. Arginine did reduce the viscosity of the antibody X formulation. It is notable that the Tm1 for antibody X in the trehalose/arginine formulation was substantially higher than the anti-IL6 antibody in the arginine formulation and yet the stability of the anti-IL6 antibody was much greater as indicated by the lower purity loss rate and the fact that it remained practically free from visible particles. These comparative examples show that arginine did not stabilize antibody X in the same way that the anti-IL6 antibody was stabilized. The purity loss rate of antibody X was not lower with arginine (remained the same) but arginine did result in instability with regard to particle formation.

TABLE 8

| | Ab conc | | | | Purity loss Rate at 40° C. | Visual appearance |
|---|---|---|---|---|---|---|
| Antibody | (mg/mL) | Formulation | Viscosity (cP) | Measured Tm1 (° C.) | (%/month) | at 5° C. |
| Anti-IL6 | 150 | 25 mM Histidine, | 14-19 | 60.6 | 1.2 | Pass, Practically |
| | | 150 mM Arg-HCl, | at | | | free from visible |
| | | 0.07% PS80, pH 6.0 | 23° C. | | | particles (9 months) |
| Antibody | 100 | 20 mM Histidine, | 4.2 at | 64.3 | 2.6 | Not acceptable, |
| X | | 240 mM trehalose, | 20° C. | | | visible particles |
| | | 50 mM Arg-HCl, pH | | | | observed (after |
| | | 6.2 | | | | 11 weeks) |
| Antibody | 100 | 20 mM Histidine, | 5.4 at | Not | 2.7 | Pass, Practically |
| X | | 240 mM Trehalose, | 20° C. | done | | free from visible |
| | | 50 mM Proline, pH | | | | particles |
| | | 6.2 | | | | (11 weeks) |

### Example 8

### Impact of Arginine and Other Excipients on the Stability of Four Different Antibodies

Experiments to assess the impact of various excipients on the stability of the anti-IL6 antibody and also several different non-anti-IL6 antibodies performed at multiple concentrations. The excipients studied were the base buffer (with no excipients), trehalose, salt, and arginine hydrochloride. The thermal stability was determined using DSC for the various antibodies. The antibody formulations were stored at 40° C. and the purity loss rate was measured using HP-SEC. High performance Size Exclusion Chromatography (HP-SEC) was performed as described in Example 1 using a TSK-GEL G3000SWXL column and SW guard column (Tosh Bioscience LLC, Mongomeryville, Pa.) with UV detection at 280 nm.

The results of the studies are summarized in Table 9. The impact of arginine compared to the base case of buffer only for all the antibodies is summarized in Table 10. There was no consistent trend in the impact of arginine on the purity loss rates for the four antibodies even though arginine did cause a reduction in the Tm1 for all the antibodies. The anti-IL6 antibody was the only antibody to be substantially stabilized by arginine out of these four antibodies. Arginine had no impact on the purity loss rate for two of the antibodies (within the assay variability about 0.2% per month purity loss difference or less). One antibody was destabilized by arginine (antibody B, Table 9, row 14).

For the anti-IL6 antibody (Table 9, rows 1-6), the arginine formulations had a lower measured Tm1 but they were the most stable when purity loss rate was assessed. In contrast, arginine decreased the Tm1 for antibody B and also increased the purity loss rate whereas trehalose increased the Tm1 and decreased the purity loss rate (Table 9, rows 11-14). For antibodies A and C the Tm1 increased for trehalose and decreased for both salt and arginine yet the purity loss rate remained with 0.2% per month (within expected variation of the assay) suggesting that all the formulations had similar stability.

TABLE 9

| | | | | | Purity loss |
|---|---|---|---|---|---|
| | | Conc | | Measured | Rate at 40° C. |
| Table Row | Antibody | (mg/mL) | Formulation | Tm1 (° C.) | (%/month) |
| 1 | anti-IL6 | 100 | 25 mM Histidine, 0.02% PS80 pH 6.0 | 63.4 | 2.3 |
| 2 | antibody | 50 | 25 mM Histidine, 225 mM | 63.8 | 3.7 |
| | | | Trehalose, 0.05% PS80, pH 6.0 | | |
| 3 | | 100 | 25 mM Histidine, 150 mM NaCl, | 61.4 | 3.0 |
| | | | 0.02% PS80, pH 6.0 | | |
| 4 | | 100 | 25 mM Histidine, 150 mM Arg-HCl, | 60.6 | 1.3 |
| | | | 0.05% PS80, pH 6.0 | | |
| 5 | | 150 | 25 mM Histidine, 150 mM Arg-HCl, | 60.6 | 1.2 |
| | | | 0.07% PS80, pH 6.0 | | |
| 6 | | 150 | 25 mM Histidine, 220 mM Arg-HCl, | 59.9 | 0.8 |
| | | | 0.07% PS80, pH 6.0 | | |
| 7 | Antibody | | 25 mM Histidine, pH 6.0 | 71.7 | 2.4 |
| 8 | A | 100 | 25 mM Histidine, 210 mM Trehalose | 72.7 | 2.4 |
| | | | pH 6.0 | | |
| 9 | | | 25 mM Histidine, 150 mM NaCl pH | 69.7 | 2.6 |
| | | | 6.0 | | |
| 10 | | | 25 mM Histidine, 150 mM Arg-HCl | 69.2 | 2.3 |
| | | | pH 6.0 | | |
| 11 | Antibody | 10 | 25 mM Histidine, pH 6.0 | 71.1 | 1.8 |
| 12 | B | | 25 mM Histidine, 210 mM Trehalose | 72.3 | 0.4 |
| | | | pH 6.0 | | |
| 13 | | | 25 mM Histidine, 150 mM NaCl pH | 68.2 | 1.6 |
| | | | 6.0 | | |
| 14 | | | 25 mM Histidine, 150 mM Arg-HCl | 67.7 | 2.5 |
| | | | pH 6.0 | | |
| 19 | Antibody | 100 | 25 mM Histidine, pH 6.0 | 62.7 | 1.0 |
| 20 | C | | 25 mM Histidine, 210 mM Trehalose | 63.9 | 0.8 |
| | | | pH 6.0 | | |
| 21 | | | 25 mM Histidine, 150 mM NaCl pH | 61.0 | 1.0 |
| | | | 6.0 | | |
| 22 | | | 25 mM Histidine, 150 mM Arg-HCl | 60.3 | 0.8 |
| | | | pH 6.0 | | |

TABLE 10

| | Impact of arginine | Impact of arginine |
|---|---|---|
| Antibody | on Tm1 | on purity loss rate |
| Anti-IL6 | Decreased Tm1 | Lower purity loss rate |
| A | Decreased Tm1 | No change in purity loss rate |
| B | Decreased Tm1 | Higher purity loss rate |
| C | Decreased Tm1 | No change in purity loss rate |

All of the various embodiments or options described herein can be combined in any and all variations. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

What is claimed is:

1. A stable, low viscosity antibody formulation comprising:
a. about 150 mg/mL to about 400 mg/mL of an anti-IL-6 antibody, and
b. greater than about 150 mM arginine,
wherein the antibody formulation is in an aqueous solution and has a viscosity of less than 20 cP at 23° C.

2. The antibody formulation of claim 1, wherein the anti-IL-6 antibody comprises a variable heavy domain (VH) and a variable light domain (VL), wherein the VH domain comprises complementarity determining regions (CDRs) comprising SEQ ID NOs: 7, 8 and 9 and the VL domain comprises CDRs comprising SEQ ID NOs. 10, 11 and 12.

3. The antibody formulation of claim 2, wherein the anti-IL-6 antibody comprises SEQ ID NO:1 and SEQ ID NO:2.

4. The antibody formation of claims 1-3, wherein the antibody is stable at 2° C. to 8° C. for 12 months as determined by SEC HPLC.

5. The antibody formulation of claims 1-3, wherein the viscosity of the antibody formulation is less than 14 cP at 23° C.

6. The antibody formulation of claims 1-3, comprising greater than 200 mM arginine.

7. The antibody formulation of claims 1-3, comprising greater than 220 mM arginine.

8. The antibody formulation of claims 1-3, comprising 150 mM to 400 mM arginine.

9. The antibody formulation of claims 1-3, further comprising a surfactant.

10. The antibody formulation of claim 7, wherein the surfactant is selected from the group consisting of polysorbate, pluronics, Brij, and other nonionic surfactants.

11. The antibody formulation of claim 8, wherein the surfactant is polysorbate 80.

12. The antibody formulation of claims 1-3, wherein the formulation further comprises histidine.

13. The antibody formulation of claims 1-3, wherein the formulation is substantially free of trehalose.

14. The antibody formulation of claims 1-3, wherein the formulation is substantially free of a disaccharide.

15. The antibody formulation of claims 1-3, wherein the formulation is substantially free of a reducing sugar, a non-reducing sugar, or a sugar alcohol.

16. The antibody formulation of claims 1-3, wherein the formulation is substantially free of an osmolyte.

17. The antibody formulation of claims 1-3, wherein the formulation has an injection force of less than 8 N when passed through a 27 Ga thin wall PFS needle.

18. The antibody formulation of claims 1-3, wherein the formulation has an osmolarity of between 300 and 450 mosm/kg.

19. The antibody formulation of claims 1-3, wherein the antibody is greater than 90% (w/w) of total polypeptide composition of the antibody formulation.

20. A stable, low viscosity antibody formulation comprising:
a. about 150 mg/mL to about 400 mg/mL of an antibody, wherein the antibody comprises amino acid sequences of SEQ ID NOS:1 and 2,
b. about 150 mM to about 400 mM arginine,
c. about 0.01% to about 0.1% polysorbate 80, and
d. about 20 mM to about 30 mM histidine,
wherein the antibody formulation has a viscosity of less than 20 cP at 23° C.

21. A stable, low viscosity antibody formulation comprising:
a. about 150 mg/mL to about 400 mg/mL of an antibody, wherein the antibody comprises a variable heavy domain (VH) and a variable light domain (VL), wherein the VH domain comprises complementarity determining regions (CDRs) comprising SEQ ID NOs: 7, 8 and 9 and the VL domain comprises CDRs comprising SEQ ID NOs. 10, 11 and 12,
b. about 150 mM to about 400 mM arginine,
c. about 0.01% to about 0.1% polysorbate 80, and
d. about 20 mM to about 30 mM histidine,
wherein the antibody formulation has a viscosity of less than 20 cP at 23° C.

22. A stable, low viscosity antibody formulation comprising:
a. about 150 mg/mL of an antibody, wherein the antibody comprises a variable heavy domain (VH) and a variable light domain (VL), wherein the VH domain comprises complementarity determining regions (CDRs) comprising SEQ ID NOs: 7, 8 and 9 and the VL domain comprises CDRs comprising SEQ ID NOs. 10, 11 and 12,
b. about 220 mM arginine,
c. about 0.07% polysorbate 80, and
d. about 25 mM histidine,
wherein the antibody formulation has a viscosity of less than 20 cP at 23° C.

23. A stable, low viscosity antibody formulation comprising:
a. about 150 mg/mL of an antibody, wherein the antibody comprises a variable heavy domain (VH) and a variable light domain (VL), wherein the VH domain comprises complementarity determining regions (CDRs) comprising SEQ ID NOs: 7, 8 and 9 and the VL domain comprises CDRs comprising SEQ ID NOs. 10, 11 and 12,
b. about 150 mM arginine,
c. about 0.07% polysorbate 80, and
d. about 25 mM histidine,
wherein the antibody formulation has a viscosity of less than 20 cP at 23° C.

24. A stable, low viscosity antibody formulation comprising:
a. about 50 mg/mL to about 200 mg/mL of an antibody, wherein the antibody comprises a variable heavy domain (VH) and a variable light domain (VL), wherein the VH domain comprises complementarity determining regions (CDRs) comprising SEQ ID NOs: 7, 8 and 9 and the VL domain comprises CDRs comprising SEQ ID NOs. 10, 11 and 12,
b. about 20 mM to about 400 mM arginine,
c. about 0.01% to about 0.1% polysorbate 80,
d. about 5 mM to about 100 mM histidine, and optionally
e. about 50 mM to about 400 mM trehalose,
wherein the antibody formulation has a viscosity of less than 20 cP at 23° C.

25. A stable, low viscosity antibody formulation comprising:
a. about 50 mg/mL of an antibody, wherein the antibody comprises a variable heavy domain (VH) and a variable light domain (VL), wherein the VH domain comprises complementarity determining regions (CDRs) comprising SEQ ID NOs: 7, 8 and 9 and the VL domain comprises CDRs comprising SEQ ID NOs. 10, 11 and 12,
b. about 0.05% polysorbate 80,
c. about 25 mM histidine, and
d. about 225 mM trehalose,
wherein the antibody formulation has a viscosity of less than 20 cP at 23° C.

26. A stable, low viscosity antibody formulation comprising:
a. about 100 mg/mL of an antibody, wherein the antibody comprises a variable heavy domain (VH) and a variable light domain (VL), wherein the VH domain comprises complementarity determining regions (CDRs) comprising SEQ ID NOs: 7, 8 and 9 and the VL domain comprises CDRs comprising SEQ ID NOs. 10, 11 and 12,
b. about 25 mM arginine,
c. about 0.07% polysorbate 80,
d. about 25 mM histidine, and
e. about 180 mM trehalose,
wherein the antibody formulation has a viscosity of less than 20 cP at 23° C.

27. A method of treating pain associated with osteoarthritis in a subject, the method comprising administering the antibody formulation of any one of claims 1-3 and 20-26.

28. A method of treating pain associated with chronic lower back pain in a subject, the method comprising administering the antibody formulation of any one of claims 1-3 and 20-26.

29. A method of treating rheumatoid arthritis in a subject, the method comprising administering the antibody formulation of any one of claims 1-3 and 20-26.

30. A method of making a stable, low viscosity antibody formulation, the method comprising:
a. concentrating an antibody to about 150 mg/mL to about 400 mg/mL, wherein the antibody comprises amino acid sequences of SEQ ID NOS:1 and 2;
b. adding arginine to the antibody of (a) to achieve an antibody formulation having a concentration of arginine of greater than about 150 mM,
wherein the antibody formulation of (b) is in an aqueous solution and has a viscosity of less than 20 cP at 23° C., and wherein the antibody formulation of (b) is stable at 2° C. to 8° C. for 12 months as determined by SEC HPLC.


SEQUENCE LISTING (12 sequences)

SEQ ID NO: 1 - protein, 450 aa, Artificial Sequence
  Description of Artificial Sequence Synthetic polypeptide
EVQLVESGGGLVQPGGSLRLSCAASGFTISSNYMIWVRQAPGKGLEWVSDLYYYAGDTYYADSVKGRFTMSRDISKNTVYLQMNSLRAEDTAVYYCARWADDHPPWIDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, Artificial Sequence
  Description of Artificial Sequence Synthetic polypeptide
DIQMTQSPSTLSASVGDRVTITCRASQGISSWLAWYQQKPGKAPKVLIYKASTLESGVPSRFSGSGSGTEFTLTISSLQPDDFATYYCQQSWLGGSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 450 aa, Artificial Sequence
  Description of Artificial Sequence Synthetic polypeptide
EVQLVESGGGLVQPGGSLRLSCAASGFTISSNYMIWVRQAPGKGLEWVSDLYYYAGDTYYADSVKGRFTMSRDISKNTVYLQMNSLRAEDTAVYYCARWADDHPPWIDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 213 aa, Artificial Sequence; identical to SEQ ID NO: 2
  Description of Artificial Sequence Synthetic polypeptide

SEQ ID NO: 5 - protein, 120 aa, Artificial Sequence
  Description of Artificial Sequence Synthetic polypeptide
EVQLVESGGGLVQPGGSLRLSCAASGFTISSNYMIWVRQAPGKGLEWVSDLYYYAGDTYYADSVKGRFTMSRDISKNTVYLQMNSLRAEDTAVYYCARWADDHPPWIDLWGRGTLVTVSS

SEQ ID NO: 6 - protein, 106 aa, Artificial Sequence
  Description of Artificial Sequence Synthetic polypeptide
DIQMTQSPSTLSASVGDRVTITCRASQGISSWLAWYQQKPGKAPKVLIYKASTLESGVPSRFSGSGSGTEFTLTISSLQPDDFATYYCQQSWLGGSFGQGTKLEIK

SEQ ID NO: 7 - protein, 5 aa, Artificial Sequence
  Description of Artificial Sequence Synthetic peptide
SNYMI

SEQ ID NO: 8 - protein, 17 aa, Artificial Sequence
  Description of Artificial Sequence Synthetic peptide
DLYYYAGDTYYADSVKG

SEQ ID NO: 9 - protein, 11 aa, Artificial Sequence
  Description of Artificial Sequence Synthetic peptide
WADDHPPWIDL

SEQ ID NO: 10 - protein, 11 aa, Artificial Sequence
  Description of Artificial Sequence Synthetic peptide
RASQGISSWLA

SEQ ID NO: 11 - protein, 7 aa, Artificial Sequence
  Description of Artificial Sequence Synthetic peptide
KASTLES

SEQ ID NO: 12 - protein, 8 aa, Artificial Sequence
  Description of Artificial Sequence Synthetic peptide
QQSWLGGS